SEQUENCE LISTING (1 sequence)

SEQ ID NO: 1 - protein, 920 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

(19) United States
(12) Patent Application Publication
(10) Pub. No.: US 2024/0150301 A1
(43) Pub. Date: May 9, 2024

(54) METHODS OF MODULATING ANDROGEN RECEPTOR CONDENSATES

(71) Applicant: ETERN BIOPHARMA (SHANGHAI) CO., LTD., Shanghai (CN)

(72) Inventors: Jingjing XIE, Shanghai (CN); Guangya ZHU, Shanghai (CN); Jidong ZHU, Shanghai (CN); Guo LI, Shanghai (CN); Hao HE, Shanghai (CN); Qiangang ZHENG, Shanghai (CN); Xiangqing JIANG, Shanghai (CN); Jing LI, Shanghai (CN); Zhenting GAO, Shanghai (CN)

(21) Appl. No.: 18/264,765

(22) PCT Filed: Feb. 10, 2022
(86) PCT No.: PCT/CN2022/075804

§ 371 Date: Aug. 9, 2023

(30) Foreign Application Priority Data
Feb. 10, 2021 (WO) PCT/CN2021/076556

(51) Int. Cl. C07D 239/42; A61K 45/06; A61P 35/00; C07D 405/12; C07K 14/72; G01N 33/50; G01N 33/68

(52) U.S. Cl. CPC C07D 239/42; A61K 45/06; A61P 35/00; C07D 405/12; C07K 14/721; G01N 33/5011; G01N 33/6845

(57) ABSTRACT

Provided are modulators of androgen receptor (AR) condensates, methods of modulating or inhibiting AR condensates, and methods of treating diseases and conditions using such modulators or inhibitors of AR condensates.

### FIELD OF THE INVENTION

The present disclosure generally relates to methods of treating androgen receptor (AR) condensates, modulators of AR condensates, and therapeutic uses thereof.

### BACKGROUND

Prostate cancer is one of the most common types of cancer in males. At its early stage, growth of prostate cancer generally depends on androgen signaling, which mediates its effects through the androgen receptor (AR). AR is a transcriptional factor that regulates expression of hundreds of genes including those involved in tumor cell growth and proliferation. Inhibitors of AR have been developed and have been found useful in treating prostate cancer. However, most of the AR inhibitors bind to the ligand binding domain, and are frequently rendered ineffective due to development of resistance.

Therefore, there exists significant needs for AR modulators with new mechanism of action.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

In one aspect, the present disclosure provides a method of treating an androgen receptor (AR)-associated disease or condition in a subject, comprising administering to the subject a therapeutically effective amount of an AR condensate modulator, wherein the AR condensate modulator modulates an AR condensate comprising at least AR.

In some embodiments, the AR is a wild type AR or an AR variant.

In some embodiments, the AR variant is resistant to at least one androgen-deprivation therapy.

In some embodiments, the AR variant comprises one or more mutations, optionally in ligand binding domain (LBD).

In some embodiments, the one or more mutations are found at the residues selected from the group consisting of L702, V716, V731, W742, H875, F877, T878, D880, L882, S889, D891, E894 μM896, E898, and T919, wherein the numbering is relative to SEQ ID NO: 1.

In some embodiments, the one or more mutations are selected from the group consisting of L702H, V716M, V731M, W742L/C, H875Y/Q, F877L, T878A/S, D880E, L882I, S889G, D891H, E894K, M896V/T, E898G, and T919S.

In some embodiments, the AR variant lacks all or part of LBD.

In some embodiments, the AR condensate is a transcriptional condensate.

In some embodiments, the wherein the transcriptional AR condensate further comprises a DNA/RNA sequence, histone, cofactor, mediator, RNA polymerase, or any combination thereof.

In some embodiments, the DNA/RNA sequence comprises AR-regulated gene, the histone comprises K27 acetylated H3, the cofactor comprises an LXXLL motif-containing protein, the mediator comprises MED1, and/or the RNA polymerase comprises phosphorylated RNA pol II.

In some embodiments, the AR condensate modulator modulates formation, stability, or activity of the AR condensate.

In some embodiments, the AR condensate modulator is an AR condensate inhibitor.

In some embodiments, the AR condensate inhibitor decreases level of the AR condensate as measured by live cell imaging. In some embodiments, level of the AR condensate is measured with stimulation by DHT. In some embodiments, the AR condensate comprises a constitutive AR variant, and the level of the AR condensate is measured without stimulation by DHT. In some embodiments, the AR condensate inhibitor decreases level of the AR condensate comprising a constitutive AR variant, as measured by live cell imaging.

In some embodiments, the level of AR condensate stimulated by DHT is quantified based on variation of fluorescence intensity within cell nucleus.

In some embodiments, the AR condensate inhibitor decreases the level of AR condensate stimulated by DHT by at least 10%, 20%, 30%, 40%, 50%, 60%, 70%, or 80%.

In some embodiments, the AR condensate inhibitor decrease expression level of at least one (or at least two, three, or four) AR-regulated gene as measured by a quantitative PCR assay in an AR-expressing cell line.

In some embodiments, the AR-regulated gene is an AR target gene or an AR variant target gene.

In some embodiments, the AR target gene is selected from the group consisting of FKBP5, KLK2, PSA, TMPRSS2 and NKX3.1

In some embodiments, the AR variant target gene is selected from the group consisting of BUB1B, CCNA2, UBE2C, KIF15, and CDC20.

In some embodiments, the AR condensate inhibitor is at least 2-fold (e.g. 3-fold, 4-fold, 5-fold, 6-fold etc.) more potent than enzalutamide at a comparable concentration in decreasing the expression level of the at least one (or at least two, three, or four) AR-regulated gene (e.g. AR target gene or AR variant target gene).

In some embodiments, the AR condensate inhibitor at a concentration of 5 μM decrease the expression level of the at least one (or at least two, three, or four) AR-regulated gene by at least 30% (e.g. at least 40%, or 50%, or 60%, or 70%), optionally as measured by quantitative PCR. In some embodiments, the AR condensate inhibitor at a concentration of 5 μM decrease the expression level of the at least one (or at least two, three, or four) AR variant target gene by at least 30% (e.g. at least 40%, or 50%, or 60%, or 70%), optionally as measured by quantitative PCR.

In some embodiments, the AR condensate inhibitor inhibits proliferation of AR-expressing cancer cells at an IC₅₀ of no more than 20 μM, or 15 μM, or 10 μM or 8 μM or 5 μM or 4 μM or 3 μM, as measured by a cell proliferation assay. In certain embodiments, the AR-expressing cancer cells express an AR variant, optionally a constitutively active AR variant (e.g. an AR variant lacking part or all of LBD).

In some embodiments, the AR-expressing cancer cells are selected from the group consisting of LNCaP, 22Rv1, VCaP and LNCaP95.

In some embodiments, the AR condensate modulator:
a) comprises Compound 60, Compound 61, Compound 62, Compound 6, Compound 2, or a pharmaceutically acceptable salt, solvate, prodrug, enantiomer, diastereomer, tautomer, isotopic substitution, polymorph or metabolite thereof, or
b) competes with Compound 60, Compound 61, Compound 62, Compound 6, or Compound 2 for binding to AR, or
c) induces a conformational change in AR at least comparable to that induced by Compound 60, Compound 61, Compound 62, Compound 6, or Compound 2;
d) has an activity comparable to or higher than that of Compound 60, Compound 61, Compound 62, Compound 6, or Compound 2 in any one or any combination of the following:
   i. decreasing level of the AR condensate, optionally stimulated by DHT;
   ii. decreasing level of the AR condensate comprising AR variant, optionally a constitutively active AR variant (e.g. an AR variant lacking part or all of LBD);
   iii. decreasing expression level of at least one (or at least two, three, or four) AR-regulated gene (e.g. AR target gene or AR variant target gene); and/or
   iv. inhibiting proliferation of AR-expressing cancer cells.

In some embodiments, the AR condensate modulator binds to the intrinsic disorder domain (IDD) of AR.

In some embodiments, the AR condensate modulator comprises a peptide, nucleic acid, or small molecule.

In some embodiments, wherein the AR-associated disease or condition is characterized in having an abnormal level of AR activity.

In some embodiments, the AR-associated disease or condition is characterized in having an elevated level of AR activity.

In some embodiments, the AR-associated disease or condition is characterized having one or more genetic aberrations that results in elevated AR activity, constitutive AR activation, or resistance to castration or androgen deprivation therapy.

In some embodiments, the one or more genetic aberrations comprise amplification of AR gene, mutations in AR gene, aberrant splicing of AR gene, rearrangement in AR gene, polymorphism in AR gene, or any combination thereof.

In some embodiments, the AR-associated disease or condition is characterized having an AR variant, optionally, the AR variant lacks all or part of the ligand binding domain (LBD).

In some embodiments, the AR-associated disease or condition is an AR-expressing cancer.

In some embodiments, the AR-expressing cancer is prostate cancer, breast cancer, glioblastoma, melanoma, bladder cancer, renal cell carcinoma, pancreatic cancer, hepatocellular carcinoma, ovarian cancer, endometrial cancer, mantle cell lymphoma, or salivary gland cancer.

In some embodiments, the AR-expressing cancer is metastatic.

In some embodiments, the AR-expressing cancer is prostate cancer.

In some embodiments, the prostate cancer is resistant to castration or androgen deprivation therapy.

In another aspect, the present disclosure provides a method of modulating transcription of one or more AR-regulated genes in a cell or a subject, comprising modulating a transcriptional AR condensate comprising at least AR.

In some embodiments, the AR is a wild type AR or an AR variant.

In some embodiments, the method comprises modulating the transcriptional AR condensate by an AR condensate modulator.

In some embodiments, the AR condensate modulator modulates formation, stability, or activity of the AR condensate.

In some embodiments, the AR condensate modulator is an AR condensate inhibitor.

In some embodiments, the AR-regulated gene is an AR target gene or an AR variant target gene.

In some embodiments, the AR target gene comprises FKBP5, KLK2, PSA, TMPRSS2, NKX3.1, or any combination thereof.

In some embodiments, the AR variant target gene comprises BUB1B, CCNA2, UBE2C, KIF15, CDC20, or any combination thereof.

In some embodiments, the AR condensate modulator:
e) comprises Compound 60, Compound 61, Compound 62, Compound 6, or Compound 2, or a pharmaceutically acceptable salt, solvate, prodrug, enantiomer, diastereomer, tautomer, isotopic substitution, polymorph or metabolite thereof, or
f) competes with Compound 60, Compound 61, Compound 62, Compound 6, or Compound 2 for binding to AR, or
g) induces a conformational change in AR at least comparable to that induced by Compound 60, Compound 61, Compound 62, Compound 6, or Compound 2;
h) has an activity comparable to or higher than that of Compound 60, Compound 61, Compound 62, Compound 6, or Compound 2 in any one or any combination of the following:
   i. decreasing level of the AR condensate, optionally stimulated by DHT;
   ii. decreasing level of the AR condensate comprising AR variant, optionally a constitutively active AR variant (e.g. an AR variant lacking part or all of LBD);
   iii. decreasing expression level of at least one (or at least two, three, or four) AR-regulated gene (e.g. AR target gene or AR variant target gene); and/or
   iv. inhibiting proliferation of AR-expressing cancer cells.

In some embodiments, the AR condensate inhibitor binds to the intrinsic disorder domain (IDD) of AR.

In some embodiments, the AR condensate modulator comprises a peptide, nucleic acid, or small molecule.

In some embodiments, the cell or subject is characterized in having an abnormal level of AR activity, or an elevated level of AR activity.

In some embodiments, the cell or subject is characterized in having one or more genetic aberrations that results in elevated AR activity, constitutive AR activation, or resistance to castration or androgen deprivation therapy.

In some embodiments, the one or more genetic aberrations comprise amplification of AR gene, mutations in AR gene, aberrant splicing of AR gene, rearrangement in AR gene, polymorphism in AR gene, or any combination thereof.

In some embodiments, the cell or subject is characterized in having an AR variant, optionally, the AR variant lacks all or part of the ligand binding domain (LBD).

In another aspect, the present disclosure provides an in vitro screening system comprising AR or an intrinsic disorder domain containing fragment thereof, wherein the AR or the fragment is attached to a detectable label and is capable of forming an AR condensate.

In some embodiments, the AR is a wild type AR or an AR variant.

In some embodiments, the detectable label comprises a fluorophore, a radioisotope, a colorimetric substrate, or an antigenic epitope.

In some embodiments, the in vitro screening system comprises a cell lysate or a nuclear lysate.

In some embodiments, the in vitro screening system comprises a cell or a nucleus.

In some embodiments, the in vitro screening system further comprises a DNA/RNA sequence, histone, cofactor, mediator, RNA polymerase, or any combination thereof.

In another aspect, the present disclosure provides a synthetic AR condensate comprising at least AR or a fragment thereof comprising IDD.

In another aspect, the present disclosure provides a modified host cell expressing AR or an intrinsic disorder domain containing fragment thereof, wherein the AR or the fragment is attached to a detectable label and is capable of forming an AR condensate.

In some embodiments, the AR is a wild type AR or an AR variant.

In some embodiments, the detectable label comprises a fluorophore, a radioisotope, a colorimetric substrate, or an antigenic epitope.

In some embodiments, the modified host cell is suitable for detection of formation of the AR condensate.

In some embodiments, the host cell is a tumor cell, optionally a prostate cancer cell.

In another aspect, the present disclosure provides a method of screening for an agent that modulates an AR condensate comprising at least AR, comprising:
a) providing the AR condensate and assessing one or more physical properties or one or more biological effects of the AR condensate,
b) contacting the AR condensate with a test agent, and
c) assessing whether the test agent causes a change in the one or more physical properties or one or more biological effects of the AR condensate.

In some embodiments, the test agent is identified as modulating the condensate if it causes a change in the one or more physical properties or one or more biological effects of the AR condensate.

In some embodiments, the physical properties comprises formation, composition, stability, and/or activity of the AR condensate.

In some embodiments, the AR condensate is contained in an in vitro screening system provided herein, in a synthetic AR condensate provided herein, or in a modified host cell provided herein.

In some embodiments, the AR condensate is an isolated synthetic condensate, or is in the form of an isolated cellular composition comprising the AR condensate.

In some embodiments, the AR condensate is inside a cell or inside nucleus.

In another aspect, the present disclosure provides a method of identifying an agent that modulates formation of an AR condensate comprising at least AR, comprising:
a) providing components capable of forming the AR condensate;
b) contacting the components with a test agent under the condition suitable for formation of the AR condensate, and
c) assessing whether presence of the test agent affects formation of the AR condensate or one or more biological effects of the AR condensate.

In some embodiments, the test agent is identified as modulating the formation of the condensate if it affects formation of the AR condensate or affects the one or more biological effects of the AR condensate.

In some embodiments, the components capable of forming the AR condensate are contained in an in vitro screening system provided herein, in a synthetic AR condensate provided herein, or in a modified host cell provided herein.

In some embodiments, the AR condensate is a transcriptional condensate.

In some embodiments, the one or more biological effects of the transcriptional condensate is assessed based on expression of an AR-regulated gene or cell proliferation.

In some embodiments, the AR-regulated gene is an AR target gene, an AR variant target gene, or a reporter gene.

In some embodiments, the test agent has been identified as capable of binding to IDD of AR.

In some embodiments, the present disclosure provides a compound selected from the group consisting of Compound 60, Compound 61, Compound 62, Compound 6, or Compound 2, or a pharmaceutically acceptable salt, solvate, prodrug, enantiomer, diastereomer, tautomer, isotopic substitution, polymorph or metabolite thereof.

In some embodiments, the present disclosure provides a pharmaceutical composition comprising a compound selected from the group consisting of Compound 60, Compound 61, Compound 62, Compound 6, or Compound 2, or a pharmaceutically acceptable salt, solvate, prodrug, enantiomer, diastereomer, tautomer, isotopic substitution, polymorph or metabolite thereof.

In some embodiments, the present disclosure provides a method of treating an AR-associated disease or condition in a subject, comprising administering to the subject a therapeutically effective amount of a compound selected from the group consisting of Compound 60, Compound 61, Compound 62, Compound 6, or Compound 2, or a pharmaceutically acceptable salt, solvate, prodrug, enantiomer, diastereomer, tautomer, isotopic substitution, polymorph or metabolite thereof. The AR-associated disease or condition is as described in the present disclosure. In some embodiment, the compound is administered in combination with a second active ingredient or therapy.

### BRIEF DESCRIPTION OF THE DRAWINGS

The accompanying drawings, which are incorporated herein, form part of the specification. Together with this written description, the drawings further serve to explain the principles of, and to enable a person skilled in the relevant art(s), to make and use the present disclosure.

FIG. 1 illustrates transcriptionally active AR formed discrete nuclear puncta. FIG. 1A: LNCaP cells stably transfected with AR-mEGFP were grown in steroid hormone depleted medium for 48 h (−DHT, left) and then stimulated with 1 nM DHT for 2 h (+DHT, right). White dashed circles indicate the nuclei. FIG. 1B: LNCaP cells transfected with AR splice variant AR V7-mEGFP were subjected to microscopic imaging 8 hours after transfection. FIG. 1C: LNCaP cells transfected with AR splice variant AR V567es-mEGFP were subjected to microscopic imaging 8 hours after transfection.

FIG. 2 illustrates activated androgen receptor (AR) nuclear puncta exhibited liquid-like properties. Representative images of the FRAP experiments with AR-mEGFP in LNCaP cells after DHT stimulation. The white arrow highlights the punctum undergoing targeted bleaching.

FIG. 3 illustrates AR forms LLPS condensates that are sites of active transcription. Co-localization of super-enhancer marker MED1 (green), actively transcribed chromatin mark H3K27ac (green) and the active RNA polymerase II phosphorylated at Ser5 of its CTD (green) with AR-mScarlet (red) puncta in H1299 cells. H1299 cells transfected with AR-mScarlet were subjected to immunofluorescence experiments using antibodies recognizing MED1, H3K27ac and Pol II S5P. Co-localization (yellow) was analyzed by Fiji.

FIG. 4 illustrates intrinsically disordered AR-NTD is responsible for driving AR phase separation. FIG. 4A illustrates graphical representation of the predicted IDRs of AR protein. PONDR (Predictor of Natural Disordered Regions) VSL2 scores are shown on the Y axis and amino acid positions on the X axis. Dashed box highlights the N-terminal domain of AR protein. FIG. 4B illustrates live-cell imaging of AR (Δ NTD)-mEGFP in LNCaP cells stimulated with or without DHT. FIG. 4C illustrates images of blue-light induced clustering of AR(NTD), AR(DBD) and AR(LBD)-CRY2-SV40 NLS at different time points in LNCaP cells. Cells were stimulated with 488 nm laser.

FIG. 5A illustrates that enzalutamide suppressed AR LLPS by inhibiting androgen binding and had no any inhibition effect on F877/T878A mutation and V7 variant. Constitutive active splice variant AR V7 displayed nuclear puncta distribution without DHT stimulation. Enzalutamide which inhibits androgen binding to AR have no effects on the phase separation of AR.

FIG. 5B illustrates Compound 60 impaired the LLPS of Enzalutamide resistant mutant and V7 variant. Constitutive active splice variant AR V7 displayed nuclear puncta distribution without DHT stimulation. Compound 60 significantly impaired the LLPS of AR V7.

FIG. 6A to 6C illustrates that Compound AA, Compound 2 and Compound 6 inhibited mRNA expression of target genes of either AR or AR-V7.

FIG. 7A to 7C illustrates that the Compound 6 and Compound 60 inhibited AR phase separation, but Compound AA did not show inhibition.

FIG. 8 shows STD NMR results of Compound 62 with purified AF-1 recombinant protein, indicating that Compound 62 binds to AF-1.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

As used herein, the term “a,” “an,” “the” and similar terms used in the context of the present invention (especially in the context of the claims) are to be construed to cover both the singular and plural unless otherwise indicated herein or clearly contradicted by the context.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In some embodiments, the agent is selected from the group consisting of a nucleic acid, a small molecule, a polypeptide, and a peptide. In certain embodiments, agents are small molecule having a chemical moiety. In some embodiments, the agent is sufficiently small to diffuse into a condensate. In some embodiments, the agent is less than about 4.4 kDa.

The terms “increased,” “increase” or “enhance” may be, for example, increase or enhancement by a statically significant amount. In some instances, for example, an element can be increased or enhanced by at least about 10% as compared to a reference level (e.g., a control), at least about 20%, at least about 30%, at least about 40%, at least about 50%, at least about 60%, at least about 70%, at least about 80%, at least about 90%, or at least about 100%, and these ranges will be understood to include any integer amount therein (e.g., 2%, 14%, 28%, etc.) which are not exhaustively listed for brevity. In other instances an element can be increased or enhanced by at least about 2-fold, at least about 3-fold, at least about 4-fold, at least about 5-fold at least about 10-fold or more as compared to a reference level.

The terms “decrease,” “reduce,” “reduced,” “reduction,” and “inhibit” may be, for example, a decrease or reduction by a statistically significant amount relative to a reference (e.g., a control). In some instances an element can be, for example, decreased or reduced by at least 10% as compared to a reference level, by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the element as compared to a reference level. These ranges will be understood to include any integer amount therein (e.g., 6%, 18%, 26%, etc.) which are not exhaustively listed for brevity.

The terms “polynucleotide” or “nucleic acid” or “oligonucleotide” are used interchangeably, and refer to a chain of covalently linked nucleotides. The nucleotides may be deoxyribonucleotides or ribonucleotides, and modified or unmodified independent from one another.

The terms “polypeptide” and “protein” are used interchangeably, and refer to a chain of amino acid residues covalently linked by peptide bonds. Proteins or polypeptide may include moieties other than amino acids (e.g., may be glycoproteins) and/or may be otherwise processed or modified. Those of ordinary skill will further appreciate that a protein can sometimes include more than one polypeptide chain, for example linked by one or more disulfide bonds or associated by other means.

The term “fragment” as used herein refers to partial sequence of the reference polypeptide or polynucleotide of any length. A fragment can still retain at least partial biological activities of the reference polypeptide.

The terms “variant” refers to a polypeptide having one or more amino acid residue changes or modification relative to a naturally occurring polypeptide.

“Functional equivalent” as used herein refers to a fragment, variant, or a fusion polypeptide of a naturally-occurring polypeptide (e.g., AR) that despite of having differences in their chemical structures retains at least partially biological functions of naturally-occurring polypeptide. In some embodiments, a functional equivalent retains at least 80%, 85%, 90%, 91%, 92%, 93%, 94%, 95%, 96%, 97%, 98%, 99% or 100% biological activity of naturally-occurring polypeptide.

The term “homologue” and “homologous” as used herein are interchangeable and refer to nucleic acid sequences (or its complementary strand) or amino acid sequences that have sequence identity of at least 80% (e.g., at least 85%, 88%, 90%, 91%, 92%, 93%, 94%, 95%, 96%, 97%, 98%, or 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “tumor” or “cancer” are used interchangeably and refers to any diseases involving an abnormal cell growth and include all stages and all forms of the disease that affects any tissue, organ or cell in the body. The term includes all known cancers and neoplastic conditions, whether characterized as malignant, benign, soft tissue, solid, or hematologic, or of all stages and grades, including pre- and post-metastatic tumors. In general, cancers can be categorized according to the tissue or organ from which the tumor is located or originated and morphology of cancerous tissues and cells.

Androgen Receptor (AR) Condensate

Androgen receptor (AR) is a member of the steroid and nuclear receptor superfamily, and is mainly expressed in androgen target tissues, such as the prostate, skeletal muscle, liver, and central nervous system (CNS), with the highest expression level observed in the prostate, adrenal gland, and epididymis. AR is a soluble protein that functions as an intracellular transcriptional factor. Upon binding and activation by androgens such as testosterone and dihydrotestosterone (DHT), AR undergoes conformational changes and posttranslational modifications, dimerization, nuclear translocation, and ultimately, binding to the regulatory regions of the DNA of target genes, known as androgen response elements.

The present disclosure surprisingly found that AR is capable of undergoing phase separation to form a condensate. “Condensate”, as used herein, refers to non-membrane-encapsulated compartment formed by phase separation (including all stages of phase separation) of one or more of proteins and/or other macromolecules (such as RNA and/or DNA) based on their intrinsic physical properties. Condensates behave as phase-separated liquids, resulting in specific proteins and/or macromolecules being concentrated inside the condensates while other specific proteins and/or macromolecules are excluded. Condensates are liquid and reversible. Upon changes in cell physiology, such as a signaling event or a change in concentration of one of the macromolecules or other changes in the local environment, the condensates within the cell will change, altering their molecular composition or even completely dissolving, thereby modulating the biological activities associated with the condensates. Biomolecular condensates mediated by phase separation form coherent structures that can compartmentalize and concentrate biochemical reactions within cells. In some embodiments, AR undergoes phase separation both in vitro and in cells, to form condensates in which AR is highly concentrated.

Wild-type AR contains three distinct domains, including an androgen-independent N-terminal domain (NTD), a DNA-binding domain, and an androgen-dependent ligand-binding domain (LBD). The full length wild type AR has an amino acid sequence of SEQ ID NO: 1, and NTD in the full length wild type AR spans from the first 559 amino acid residues. NTD of AR contains high degree of intrinsic disorder, with few alpha-helices and beta-sheets. The disordered region that lack a fixed or ordered secondary and tertiary structure in a protein is also referred to as intrinsic disordered regions (IDD). IDDs can range from fully unstructured to partially structured. In some embodiments, an IDD may be identified by the methods disclosed in Ali, M., & Ivarsson, Y. (2018). High-throughput discovery of functional disordered regions. Molecular Systems Biology, 14(5), e8377.

NTD of AR is found to contain extensive IDDs. Without bound to any theory, phase separation or condensate formation of AR is believed to be driven by multivalent interaction via adhesions or associations mediated by IDD.

In addition, NTD of AR is known to contain the activation function-1 (AF1) region, which is essential for AR transactivation and is present in different forms of the AR variants.

The term “androgen receptor” or “AR” as used herein encompasses both wild type AR or AR variant. Wild type AR as used herein refers to the full length AR, having an exemplary sequence of SEQ ID NO: 1. AR variant as used herein encompasses all different forms of AR that retains at least part of the NTD and can phase separate or can form condensates. Examples of AR variants include, without limitation, mutants, fragments, fusions, and splicing variants.

In some embodiments, the AR variant is resistant to at least one androgen-deprivation therapy. The term “androgen deprivation therapy” or “ADT” as used herein refers to therapies that suppress androgen, by reducing levels of androgen or by inhibiting biological functions of androgen such as by inhibiting AR signaling. ADT can include both surgical treatments (such as surgical castration) and drug treatments. Examples of ADT drugs include, without limitation, LHRH agonists (such as Leuprolide (Lupron, Eligard), Goserelin (Zoladex), Triptorelin (Trelstar), and Histrelin (Vantas)), LHRH antagonists (such as Degarelix (Firmagon), Relugolix (Orgovyx)), drugs that lower androgen levels from the adrenal glands (such as Abiraterone (Zytiga), Ketoconazole (Nizoral)), androgen receptor antagonists (such as Flutamide (Eulexin), Bicalutamide (Casodex), Nilutamide (Nilandron)), and other anti-androgens (such as Enzalutamide (Xtandi), apalutamide (Erleada) and darolutamide (Nubega)).

In some embodiments, the AR variant comprises one or more mutations. Over 800 different mutations have been identified with AR in patients with androgen insensitivity syndrome, and prostate cancer. In the AR gene, four different types of mutations have been detected to inactivate AR, including: a) single point mutations resulting in amino acid substitutions or premature stop codons; b) nucleotide insertions or deletions leading to a frame shift and premature rumination; c) complete or partial gene deletions; and d) intronic mutations causing alternative splicing (see, for details, K. Eisermann et al, Transl Androl, Urol. 2013 September; 2(3): 137-147).

In some embodiments, the AR variant comprises one or more mutations in ligand binding domain (LBD). In some embodiments, the one or more mutations in the LBD results in gain of function in AR.

In some embodiments, the AR variant comprises one or more mutations at the residues selected from the group consisting of L702, V716, V731, W742, H875, F877, T878, D880, L882, S889, D891, E894 μM896, E898, and T919, wherein the numbering is relative to SEQ ID NO: 1. Amino acid sequence of SEQ ID NO: 1 is provided below (also see, NCBI accession number: NP_000035.2):

MEVQLGLGRVYPRPPSKTYRGAFQNLFQSVREVIQNPGPRHPEAASAAPP
GASLLLLQQQQQQQQQQQQQQQQQQQQQQQETSPRQQQQQQGEDGSPQAH
RRGPTGYLVLDEEQQPSQPQSALECHPERGCVPEPGAAVAASKGLPQQLP
APPDEDDSAAPSTLSLLGPTFPGLSSCSADLKDILSEASTMQLLQQQQQE
AVSEGSSSGRAREASGAPTSSKDNYLGGTSTISDNAKELCKAVSVSMGLG
VEALEHLSPGEQLRGDCMYAPLLGVPPAVRPTPCAPLAECKGSLLDDSAG
KSTEDTAEYSPFKGGYTKGLEGESLGCSGSAAAGSSGTLELPSTLSLYKS
GALDEAAAYQSRDYYNFPLALAGPPPPPPPPHPHARIKLENPLDYGSAWA
AAAAQCRYGDLASLHGAGAAGPGSGSPSAAASSSWHTLFTAEEGQLYGPC
GGGGGGGGGGGGGGGGGGGGGGGEAGAVAPYGYTRPPQGLAGQESDFTAP
DVWYPGGMVSRVPYPSPTCVKSEMGPWMDSYSGPYGDMRLETARDHVLPI
DYYFPPQKTCLICGDEASGCHYGALTCGSCKVFFKRAAEGKQKYLCASRN
DCTIDKFRRKNCPSCRLRKCYEAGMTLGARKLKKLGNLKLQEEGEASSTT
SPTEETTQKLTVSHIEGYECQPIFLNVLEAIEPGVVCAGHDNNQPDSFAA
LLSSLNELGERQLVHVVKWAKALPGFRNLHVDDQMAVIQYSWMGLMVFAM
GWRSFTNVNSRMLYFAPDLVFNEYRMHKSRMYSQCVRMRHLSQEFGWLQI
TPQEFLCMKALLLFSIIPVDGLKNQKFFDELRMNYIKELDRIIACKRKNP
TSCSRRFYQLTKLLDSVQPIARELHQFTFDLLIKSHMVSVDFPEMMAEII
SVQVPKILSGKVKPIYFHTQ

In some embodiments, the one or more mutations are selected from the group consisting of L702H, V716M, V731M, W742L/C, H875Y/Q, F877L, T878A/S, D880E, L882I, S889G, D891H, E894K, M896V/T, E898G, and T919S.

In some embodiments, the AR variant lacks all or part of LBD.

In some embodiments, the AR condensate is a transcriptional condensate. Emerging evidence has shown that gene expression is accompanied by the recruitment of large clusters of transcription complexes, such as mediator and RNA Polymerase II (Pol II) that form condensates through phase separation. “Transcriptional condensates” as used herein refers to phase-separated condensates that occur at the sites of transcription and contain multiple cooperating components related to transcription. “Component” with regard to a condensate, as used herein, refers to a molecule that can be found in or on a condensate under physiological or pathological conditions. The components in a transcriptional condensate can include transcription factors, co-factors, chromatin regulators, DNA, non-coding RNA, nascent RNA, and RNA polymerase II, and the like.

In some embodiments, the transcriptional AR condensate comprises AR, and further comprises a DNA/RNA sequence, histone, cofactor, mediator, RNA polymerase, or any combination thereof.

In some embodiments, the DNA/RNA sequence comprises AR-regulated gene.

In some embodiments, the transcriptional AR condensate further comprises a histone. Eukaryotic transcription is regulated by chromatin structure, whose alterations are mediated by conserved post-translational histone tail modifications. Histone tail modification includes, without limitation, acetylation, methylation, phosphorylation, and ubiquitination. Histone acetylation, typically catalyzed by histone acetyltransferase that acetylates the lysine residues within the histone tail, decreases the interaction of histone with DNA, thereby transforming the condensed chromatin into a more relaxed structure to facilitate greater levels of gene transcription. In some embodiments, the histone comprises H3K27ac. In some embodiments, the histone comprises K27 acetylated H3.

In some embodiments, the cofactor comprises an LXXLL motif-containing protein. LXXLL motifs are present in many transcription factors and cofactors, mediating interactions that can activate or repress transcription. Co-factors having LXXLL motifs are known in the art.

Examples of mediators include, without limitation, MED1, MED15, GCN4, p300, BRD4, a hormone (e.g. estrogen) or TFIID. In some embodiments, the mediator comprises MED1.

In some embodiments, the transcriptional AR condensate further comprises a RNA polymerase. “RNA polymerase II” or Pol II as used herein refers to a multiprotein complex of 12 subunits that transcribes DNA into pre-mRNA and most small nuclear RNA and microRNA. A wide range of transcription factors are required for Pol II to bind to upstream gene promoters and begin transcription. The synthesis of pre-mRNA by RNA polymerase II (Pol II) involves the formation of a transcription initiation complex and a transition to an elongation complex. The large subunit of Pol II contains an intrinsically disordered C-terminal domain (CTD), which is phosphorylated by cyclin-dependent kinases (CDKs) during the initiation-to-elongation transition, thus influencing the CTD's interaction with different components of the initiation.

In some embodiments, the RNA polymerase comprises phosphorylated RNA pol II.

AR Condensate Modulator

In cells, it is found by the inventors that AR condensates occupy the sites of active gene transcription. While phase separated, AR can interact with multiple components which may or may not by themselves phase separate but can reach high local concentrations in the condensate, and promote gene transcription. Therefore, modulation of transcriptional AR condensate is believed to be able to modulate transcription or expression of the AR-regulated genes.

The present disclosure also provides AR condensate modulators that modulates an AR condensate comprising at least AR.

In some embodiments, the AR condensate modulator modulates formation, stability, and/or activity of the AR condensate. In some embodiments, modulating an AR condensate also includes changing the morphology or shape of the AR condensate, and/or regulation of cell signaling cascade that involves one or more component associated with the AR condensate.

“Formation” with regard to an AR condensate, as used herein, refers to the generation of an AR condensate with well-delineated physical boundaries, but without lipid membrane barriers. In some embodiments, the formation of AR condensate can be driven by phase separation, in particular phase separation of AR. In some embodiments, disturbing the ability of AR to phase separate impairs the formation of AR condensate. In some embodiments, the formation of AR condensate is driven by phase separation of a component of the AR condensate other than AR. In some embodiments, modulating the formation of a condensate includes increasing or decreasing the rate of formation or whether or not formation occurs.

“Composition” as used herein, refers to the collection of components associated within a condensate, in particular a transcriptional condensate. An AR transcriptional condensate typically comprises multiple components, including proteins and/or nucleic acids. In some embodiments, the composition of an AR condensate is heterogeneous or dynamic. In some embodiments, the composition of an AR condensate can be modulated, for example, by increasing or decreasing the level of a component associated with the condensate.

“Stability” as used herein refers to the property of a condensate that when disturbed from a condition of equilibrium to restore its original condition. The stability of a condensate can be reflected by the maintenance or dissolution (either partial or complete) of a condensate. Maintenance refers to the preserving of the composition and physical properties of a condensate. In contrast, dissolution refers to the disassembly of a condensate, either partially or completely. In some embodiments, modulating the stability of a condensate includes increasing or decreasing the rate of condensate maintenance or dissolution, or promoting or suppressing condensate dissolution.

“Activity” as used herein refers to the activity of an AR condensate in regulating the transcription of the genes that are targeted by the AR condensate. In some embodiments, the activity of a condensate is correlated with the composition or stability of a condensate. Changes in the composition of a condensate may affect the activity of the condensate. In some embodiments, modulating condensate activity includes modifying the transcriptional activity of a condensate.

AR Condensate Inhibitor

In some embodiments, the transcriptional AR condensate is modulated by contacting with an AR condensate inhibitor. In some embodiments, the AR condensate modulator is an AR condensate inhibitor.

“AR condensate inhibitor” as used herein refers to an agent capable of inhibiting the level or activity of an AR condensate. In certain embodiments, the AR condensate inhibitor disturbs, reduces, or suppresses the formation, composition, stability, or activity of the transcriptional AR condensate.

In certain embodiments, the AR condensate inhibitor reduces the level or formation of the transcriptional AR condensate. For example, the AR condensate inhibitor may disrupt the interactions required for maintenance or formation of the AR condensate, or may induce dissolution of the AR condensate, or may cause change in composition in the AR condensate that alters or inhibits its transcriptional activity.

In some embodiments, the AR condensate modulator interacts with the intrinsic disorder domain (IDD) of AR. IDD of AR is believed to participate in formation, maintenance, dissolution or regulation of the AR condensate, and interaction with IDD can modulate the level or formation of the AR condensate.

In some embodiments, the IDD has separate discrete regions. In some embodiments, the IDD is at least about 5, 10, 15, 20, 30, 40, 50, 60, 75, 100, 150, or more disordered amino acids (e.g., contiguous disordered amino acids). In some embodiments, an amino acid is considered a disordered amino acid if at least 75% of the algorithms employed by D²P² (Oates et al., Nucleic Acids Res., 2013 January; 41(Database issue):D508-16.) predict the residue to be disordered. IDD of AR is known to be within the N terminal domain (NTD) of AR, and can be discrete regions in NTD of AR.

In some embodiments, the AR condensate modulator interacts with an IDD-containing fragment of NTD of AR. In some embodiments, IDD-containing fragment is tau-1 or tau-5. Tau-1 covers residues from amino acid positions 102 to 371 of AR, with numbering relative to SEQ ID NO: 1. Tau-5 covers residues from amino acid positions 330 to 448 of AR, with numbering reference to SEQ ID NO: 1.

In some embodiments, the IDD-containing fragment is contained within a region from amino acid positions 102 to 371, positions 102 to 300, positions 102 to 250, positions 102 to 200, positions 102 to 150, positions 150 to 300, positions 200 to 300, positions 250 to 300, with numbering relative to SEQ ID NO: 1. In some embodiments, the IDD-containing fragment is contained within a region from amino acid positions 330 to 448, positions 330 to 420, positions 330 to 400, positions 330 to 380, positions 330 to 360, positions 350 to 448, positions 380 to 448, positions 400 to 448, or positions 400 to 420, with numbering relative to SEQ ID NO: 1. In some embodiments, the IDD-containing fragment has a length of about or at least 20 residues, 25 residues, 30 residues, 40 residues, or 50 residues.

In some embodiments, the AR condensate modulator binds to a residue within an IDD-containing fragment provided herein. In some embodiments, the AR condensate modulator binds to cysteine 406 of SEQ ID NO: 1. In some embodiments, the AR condensate modulator binds to a fragment comprising cysteine 406 of SEQ ID NO: 1.

In some embodiments, the AR condensate modulator binds to the IDD of the AR, or binds to a region outside of the IDD but can allosterically affect IDD. Binding to IDD can be determined using suitable methods known in the art. In some embodiments, the region outside of the IDD is within the NTD of AR.

Binding site of the AR condensate modulator to the AR can be determined based suitable methods known in the art, for example, by Saturation Transfer Diffusion Nuclear Magnetic Resonance (STD NMR) affinity assay (such as described in Example 7), mass spectrometry analysis (such as described in Example 8) or pull down assay (such as described in Example 9).

Methods for STD NMR are described in, for example, Walpole, S. et al, Methods in Enzymology, Volume 615, 2019, Pages 423-451. For mass spectrometry, in brief, a recombinant AR protein or IDD-containing fragment of AR can be incubated with a test compound, followed by fragmentation of the protein, and mass spectrometry determination. With respect to pull down assay, a recombinant IDD-containing fragment can be contacted with a test compound, followed by enrichment of the compound/fragment complex, and determination of binding of the compound to the fragment.

Level of AR condensate can be determined by any suitable methods, including, without limitation, by live cell imaging. Live cell imaging can be performed via microscopy, for example, deconvolution microscopy, structured illumination microscopy, or interference microscopy.

In certain embodiments, the level of AR condensate can be determined by live cell imaging. For example, AR may be conjugated with a detectable label such as a fluorescent molecule, which allows AR to be visualized under microscope, and the fluorescence intensity can indicate presence and/or level of AR condensate. For example, when wild-type AR does not form AR condensate, the fluorescence intensity of the labeled AR generally appear evenly distributed in nucleus or cytosol. However, upon androgen stimulation, formation or presence of AR condensate can be observed as bright spots or puncta in contrast to background, where the bright spots or puncta indicates presence of AR condensates. However, for certain constitutively active AR variants (such as those lacking LBD domain), the AR condensates can spontaneously form in the absence of androgen stimulation, and level of AR condensates can be observed or determined using live cell imaging. The level of AR condensates can be further determined by a suitable method, for example, by counting the number of AR condensates visualized under a selected field under the microscope, or by calculating the increased fluorescence intensity signal relative to the background signal, or by calculating the area where fluorescence intensity is above a predetermined threshold.

In some embodiments, the AR condensate inhibitor decreases level of the AR condensate stimulated by androgen (such as DHT), as measured by live cell imaging. A skilled person in the art can select a suitable concentration of androgen to be used in the assay, as long as it can provide for a suitable window to determine inhibition of the AR condensate. In an example, 1 nM of DHT can be used in a cellular assay to detect AR condensate inhibition. In some embodiments, the AR condensate inhibitor, at a concentration of no more than 10 μM, decreases level of AR condensate stimulated by 1 nM DHT, as measured by live cell imaging, as described in Examples 2 and 4.

However, it should be understood that stimulation of androgen is not necessary for AR condensate formation or test of inhibition on AR condensate level, as certain AR variants are capable of forming AR condensate in the absence of an androgen such as DHT. In such cases, the AR condensate inhibitor can decrease level of the AR condensate in the absence of an androgen.

In some embodiments, the AR condensate inhibitor decreases the level of AR condensate, optionally stimulated by DHT, by at least 10%, 20%, 30%, 40%, 50%, 60%, 70%, or 80%. In some embodiments, the AR condensate inhibitor, at a concentration of no more than 10 μM, decreases the level of AR condensate, optionally stimulated by 1 nM DHT by at least 10%, 20%, 30%, 40%, 50%, 60%, 70%, or 80%, as measured by live cell imaging, as described in Examples 2 and 4.

In some embodiments, the AR condensate inhibitor decreases the level of AR condensate comprising AR variant, optionally a constitutively active AR variant (e.g. an AR variant lacking part of all of LBD), by at least 10%, 20%, 30%, 40%, 50%, 60%, 70%, or 80%. In some embodiments, the AR condensate inhibitor is at a concentration of no more than 10 μM. In some embodiments, the decrease in the level of AR condensate is measured by live cell imaging, as described in Examples 2 and 4.

In some embodiments, the level of AR condensate stimulated by DHT is quantified based on variation of fluorescence intensity within cell nucleus. Variation of fluorescence intensity can be determined under microscope.

In some embodiments, the level of AR condensate is quantified based on the number of puncta that shows increased fluorescence intensity over the background fluorescence within cell nucleus. In some embodiments, the level of AR condensate is quantified based on the area that shows increased fluorescence intensity over the background fluorescence within cell nucleus.

Alternatively, activity of AR condensate can be determined by any suitable methods, including, without limitation, by determination of expression of AR-regulated genes. Any suitable methods for determining gene expression can be used, for example, without limitation, quantitative PCR, reporter assay, western blot, and the like.

In some embodiments, the AR condensate inhibitor decrease expression level of at least one (or at least two, three, or four) AR-regulated gene as measured by a quantitative PCR assay in an AR-expressing cell line.

In some embodiments, the AR-regulated gene is an AR target gene or an AR variant target gene.

As used herein, “AR target gene” refers to genes of which transcription is regulated by wildtype AR. Examples of AR target genes include, without limitation, FKBP5, KLK2, PSA, TMPRSS2 and NKX3.1.

As used herein, “AR variant target gene” refers to genes of which transcription is specifically regulated by AR variant, but not by wildtype AR. Examples of AR variant target genes include, without limitation, BUB1B, CCNA2, UBE2C, KIF15, and CDC20. For more details, please see Hu, R et al, Cancer Res 2012; 72:3457-3462, which are incorporated herein to its entirety.

Without wishing to be bound by any theory, it is believed that the genes regulated by wildtype AR and AR variants are not the same, or are not to the same extent. In particular, certain genes regulated by AR variants are not effectively inhibited by existing AR inhibitors such as enzalutamide. However, an AR condensate inhibitor provided herein can inhibit expression of genes that are regulated by wildtype AR as well as genes that are preferentially regulated by AR variants, and in some embodiments the inhibition by the AR condensate inhibitor provided herein is significantly higher than that by existing AR inhibitors such as enzalutamide.

In some embodiments, the AR condensate inhibitor provided herein is at least 2-fold (e.g. 3-fold, 4-fold, 5-fold, 6-fold etc.) more potent than enzalutamide at a comparable concentration in decreasing the expression level of the at least one (or at least two, three, or four) AR-regulated gene. Enzalutamide is an androgen receptor inhibitor known to compete with androgens for binding to the ligand binding domain (LBD) of AR. The X fold potency, with respect to decrease in expression level of a gene, means the decrease in expression level of one or more genes is X fold of that achieved by a reference compound at a similar concentration. For example, if an AR condensate inhibitor decreases expression level of a gene by 60% at a concentration of 5 μM, and a reference compound decreases expression level of the same gene by 30% at a concentration of 5 μM, then the AR condensate inhibitor is 2 fold more potent than the reference compound.

In some embodiments, the AR condensate inhibitor provided herein is at least 2-fold (e.g. 3-fold, 4-fold, 5-fold, 6-fold etc.) more potent than enzalutamide at a comparable concentration in decreasing the expression level of two, three, four or five of the AR target genes selected from the group consisting of FKBP5, KLK2, PSA, TMPRSS2 and NKX3.1. In some embodiments, AR condensate inhibitor and enzalutamide are tested at 0.1 uM, 0.5 uM, 1 uM, 2 uM, 5 μM, 10 μM, 15 μM, or 20 μM.

In some embodiments, the AR condensate inhibitor provided herein is at least 2-fold (e.g. 3-fold, 4-fold, 5-fold, 6-fold etc.) more potent than enzalutamide at a comparable concentration in decreasing the expression level of two, three, four or five of the AR variant target genes selected from the group consisting of BUB1B, CCNA2, UBE2C, KIF15, and CDC20. In some embodiments, AR condensate inhibitor and enzalutamide are tested at 0.1 uM, 0.5 uM, 1 uM, 2 uM, 5 μM, 10 μM, 15 μM, or 20 μM.

In some embodiments, the AR condensate inhibitor at a concentration of 5 μM decrease the expression level of the at least one (or at least two, three, or four) AR-regulated gene by at least 30% (e.g. at least 40%, or 50%, or 60%, or 70%), optionally as measured by quantitative PCR.

In some embodiments, the AR condensate inhibitor provided herein inhibits proliferation of AR-expressing cancer cells at an IC₅₀ of no more than 20 μM, or 15 μM, or 10 μM, or 10 μM or 8 μM or 5 μM or 4 μM or 3 μM, optionally as measured by cell viability assay. In some embodiments, the AR-expressing cancer cells express an AR variant.

In some embodiments, the AR-expressing cancer cells are selected from the group consisting of LNCaP, 22Rv1, VCaP, and LNCaP95.

LNCaP is a human prostate carcinoma cell line that expresses wild-type human androgen receptor (AR). LNCaP is available from commercial sources such as DSMZ under accession number of ACC 256.

22Rv1 is a human prostate carcinoma epithelial cell line expressing both wild-type AR and an AR splice variant 7 (AR-V7) that lacks the ligand-binding domain. 22Rv1 is available from commercial sources such as ATCC under accession number of CRL-2505.

VCaP is a human prostate cancer cell line expressing both wild-type AR and AR-V7 which lacks the ligand-binding domain. VCaP is available from commercial sources such as ATCC under accession number of CRL-2876.

LNCaP95 is derived from the LNCaP cell line but has been cultivated under androgen-stripped serum conditions for a long time, making them resistant to androgen deprivation therapy (ADT), and they express both wild-type AR and AR-V7.

In some embodiments, the AR condensate modulator comprises a peptide, nucleic acid, or small molecule.

The term “peptide” and “polypeptide” are used interchangeably, and refer to a chain of amino acid residues covalently linked by peptide bonds. Peptide may include moieties other than amino acids (e.g., may be glycoproteins) and/or may be otherwise processed or modified. Those of ordinary skill will further appreciate that a peptide can sometimes include more than one peptide chain, for example linked by one or more disulfide bonds or associated by other means.

The terms “polynucleotide” or “nucleic acid” or “oligonucleotide” are used interchangeably, and refer to a chain of covalently linked nucleotides. The nucleotides may be deoxyribonucleotides or ribonucleotides, and modified or unmodified independent from one another.

The term “small molecule” as used herein refers to a chemical molecule such as a compound that is not a peptide or a nucleic acid. In certain embodiments, a small molecule can be less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric.

In some embodiments, the AR condensate modulator comprises Compound 60, Compound 61, Compound 62, Compound 6, Compound 2, or a pharmaceutically acceptable salt, solvate, prodrug, enantiomer, diastereomer, tautomer, isotopic substitution, polymorph or metabolite thereof.

“Compound 6” as used herein refers to 3-chloro-2-(2-chloroethoxy)-5-(2-(4-((2-((1-oxido-1λ⁶-thiomorpholin-1-ylidene) amino)pyrimidin-4-yl)methoxy)phenyl)propan-2-yl)benzonitrile.

“Compound 60” as used herein refers to N-(5-((4-(2-(3-chloro-4-(2-chloroethoxy)-5-cyanophenyl)propan-2-yl)phenyl)ethynyl)pyrimidin-2-yl)methanesulfonamide.

“Compound 61” as used herein refers to 3-chloro-5-(2-(4-((2-((1-oxidotetrahydro-1λ⁶-thiophen-1-ylidene)amino)pyrimidin-5-yl)ethynyl)phenyl)propan-2-yl)-2-(oxiran-2-ylmethoxy)benzonitrile.

“Compound 2” as used herein refers to 3-chloro-2-(2-chloroethoxy)-5-(2-(4-((2-((1-oxidotetrahydro-1λ⁶-thiophen-1-ylidene) amino) pyrimidin-4-yl) methoxy) phenyl) propan-2-yl) benzonitrile.

“Compound 62” as used herein refers to 3-chloro-2-(2-hydroxyethoxy)-5-(2-(4-((2-((1-oxido-1λ⁶-thiomorpholin-1-ylidene)amino)pyrimidin-4-yl)methoxy)phenyl)propan-2-yl)benzonitrile.

The chemical structures of Compound 6, Compound 60, Compound 2, Compound 61, and Compound 62 are shown below:

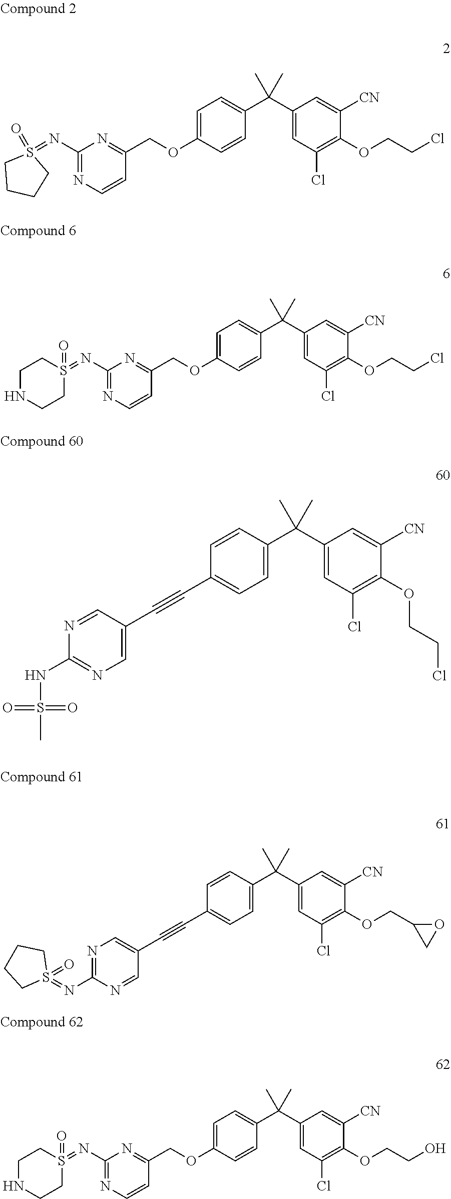 [SMILES: CC(C)(c1ccc(OCc2ccnc(N=S3(=O)CCCC3)n2)cc1)c1cc(Cl)c(OCCCl)c(C#N)c1 ; CC(C)(c1ccc(OCc2ccnc(N=S3(=O)CCNCC3)n2)cc1)c1cc(Cl)c(OCCCl)c(C#N)c1 ; CC(C)(c1ccc(C#Cc2cnc(NS(C)(=O)=O)nc2)cc1)c1cc(Cl)c(OCCCl)c(C#N)c1 ; CC(C)(c1ccc(C#Cc2cnc(N=S3(=O)CCCC3)nc2)cc1)c1cc(Cl)c(OCC2CO2)c(C#N)c1 ; CC(C)(c1ccc(OCc2ccnc(N=S3(=O)CCNCC3)n2)cc1)c1cc(Cl)c(OCCO)c(C#N)c1]

In some embodiments, the AR condensate modulator competes with Compound 60, Compound 61, Compound 62, Compound 6, or Compound 2 for binding to AR.

In some embodiments, the AR condensate modulator induces a conformational change in AR at least comparable to that induced by Compound 60, Compound 61, Compound 62, Compound 6, or Compound 2.

In some embodiments, the AR condensate modulator has an activity comparable to or higher than that of Compound 60, Compound 61, Compound 62, Compound 6, or Compound 2 in any one or any combination of the following: a) decreasing level of the AR condensate, optionally stimulated by DHT; b) decreasing expression level of at least one (or at least two, three, or four) AR-regulated gene; and/or c) inhibiting proliferation of AR-expressing cancer cells.

In some embodiments, the AR condensate modulator has an activity comparable to or higher than that of Compound 60, Compound 61, Compound 62, Compound 6, or Compound 2 in any one or any combination of the following: a) decreasing level of the AR condensate comprising an AR variant; b) decreasing expression level of at least one (or at least two, three, or four) AR-regulated gene (e.g. AR target gene or AR variant target gene); and/or c) inhibiting proliferation of AR-expressing cancer cells.

Compounds and Pharmaceutical Composition

In another aspect, the present disclosure provides a compound selected from the group consisting of Compound 60, Compound 61, Compound 62, Compound 6, or Compound 2, or a pharmaceutically acceptable salt, solvate, prodrug, enantiomer, diastereomer, tautomer, isotopic substitution, polymorph or metabolite thereof.

In another aspect, the present disclosure provides a pharmaceutical composition comprising a compound selected from the group consisting of Compound 60, Compound 61, Compound 62, Compound 6, or Compound 2, or a pharmaceutically acceptable salt, solvate, prodrug, enantiomer, diastereomer, tautomer, isotopic substitution, polymorph or metabolite thereof, and at least one pharmaceutical acceptable excipient

As used herein, the term “pharmaceutically acceptable” indicates that the substance or composition is compatible chemically and/or toxicologically, with the other ingredients comprising a formulation, and/or the subjects being treated therewith.

As used herein, the term “pharmaceutically acceptable salt”, unless otherwise indicated, includes salts that retain the biological effectiveness of the free acids and bases of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, PA, Vol. 2, p. 1457, 1995; “Handbook of Pharmaceutical Salts: Properties, Selection, and Use” by Stahl and Wermuth, Wiley-VCH, Weinheim, Germany, 2002. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. Thus, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

It is also to be understood that the compounds of present disclosure can exist in unsolvated forms, solvated forms (e.g., hydrated forms), and solid forms (e.g., crystal or polymorphic forms), and the present disclosure is intended to encompass all such forms.

As used herein, the term “solvate” or “solvated form” refers to solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. Examples of solvents that form solvates include, but are not limited to, water, isopropanol, ethanol, methanol, DMSO, ethyl acetate, acetic acid, and ethanolamine.

As used herein, the terms “crystal form”, “crystalline form”, “polymorphic forms” and “polymorphs” can be used interchangeably, and mean crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The present disclosure is also intended to include all isotopes of atoms in the compounds. Isotopes of an atom include atoms having the same atomic number but different mass numbers. For example, unless otherwise specified, hydrogen, carbon, nitrogen, oxygen, phosphorous, sulfur, fluorine, chlorine, bromide or iodine in the compounds of present disclosure are meant to also include their isotopes, such as but not limited to ¹H, ²H, ³H, ¹¹C, ¹²C, ¹³C, ¹⁴C, ¹⁴N, ⁵N, ¹⁶O, ¹⁷O, ¹⁸O, ³¹P, ³²P, ³²S, ³³S, ³⁴S, ³⁶S, ¹⁷F, ¹⁸F, ¹⁹F, ³⁵Cl, ³⁷Cl, ⁷⁹Br, ⁸¹Br, ¹²⁴I, ¹²⁷I and ¹³¹I. In some embodiments, hydrogen includes protium, deuterium and tritium. In some embodiments, carbon includes ¹²C and ¹³C.

Those of skill in the art will appreciate that compounds of the present disclosure may exist in different tautomeric forms, and all such forms are embraced within the scope of the present disclosure. The term “tautomer” or “tautomeric form” refers to structural isomers of different energies which are interconvertible via a low energy barrier. The presence and concentrations of the isomeric forms will depend on the environment the compound is found in and may be different depending upon, for example, whether the compound is a solid or is in an organic or aqueous solution. By way of examples, proton tautomers (also known as prototropic tautomers) include interconversions via migration of a proton, such as keto-enol, amide-imidic acid, lactam-lactim, imine-enamine isomerizations and annular forms where a proton can occupy two or more positions of a heterocyclic system. Valence tautomers include interconversions by reorganization of some of the bonding electrons. Tautomers can be in equilibrium or sterically locked into one form by appropriate substitution. Compounds of the present disclosure identified by name or structure as one particular tautomeric form are intended to include other tautomeric forms unless otherwise specified.

As used herein, the term “prodrugs” refers to compounds or pharmaceutically acceptable salts thereof which, when metabolized under physiological conditions or when converted by solvolysis, yield the desired active compound. Prodrugs include, without limitation, esters, amides, carbamates, carbonates, ureides, solvates, or hydrates of the active compound. Typically, the prodrug is inactive, or less active than the active compound, but may provide one or more advantageous handling, administration, and/or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound. Prodrugs may proceed from prodrug form to active form in a single step or may have one or more intermediate forms which may themselves have activity or may be inactive. Preparation and use of prodrugs is discussed in T. Higuchi and V. Stella, “Pro-drugs as Novel Delivery Systems”, Vol. 14 of the A.C.S. Symposium Series, in Bioreversible Carriers in Drug Design, ed. Edward B. Roche, American Pharmaceutical Association and Pergamon Press, 1987; in Prodrugs: Challenges and Rewards, ed. V. Stella, R. Borchardt, M. Hageman, R. Oliyai, H. Maag, J. Tilley, Springer-Verlag New York, 2007, all of which are hereby incorporated by reference in their entirety.

As used herein, the term “metabolite”, e.g., active metabolite overlaps with prodrug as described above. Thus, such metabolites are pharmacologically active compounds or compounds that further metabolize to pharmacologically active compounds that are derivatives resulting from metabolic process in the body of a subject. For example, such metabolites may result from oxidation, reduction, hydrolysis, amidation, deamidation, esterification, deesterification, enzymatic cleavage, and the like, of the administered compound or salt or prodrug. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques know in the art. See, e.g., Bertolini et al, 1997, J Med Chem 40:2011-2016; Shan et al., J Pharm Sci 86:756-757; Bagshawe, 1995, DrugDev Res 34:220-230; Wermuth, supra.

The compounds provided herein can be prepared using any known organic synthesis techniques and can be synthesized according to any of numerous possible synthetic routes, including, without limitation those described in the Examples of the present disclosure.

Reactions for preparing compounds of the present disclosure can be carried out in suitable solvents, which can be readily selected by one skilled in the art of organic synthesis. Suitable solvents can be substantially non-reactive with starting materials (reactants), intermediates, or products at the temperatures at which the reactions are carried out, e.g. temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), in P. Kocienski, Protecting Groups, Georg Thieme Verlag, 2003, and in Peter G. M. Wuts, Greene's Protective Groups in Organic Synthesis, 5^{th} Edition, Wiley, 2014, all of which are incorporated herein by reference in its entirety.

Reactions can be monitored according to any suitable method known in the art. For example, product formation can be monitored by spectroscopic means, such as nuclear magnetic resonance spectroscopy (e.g. ¹H or ¹³C), infrared spectroscopy, spectrophotometry (e.g. UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6(6), 874-883, which is incorporated herein by reference in its entirety), and normal phase silica chromatography.

In another aspect, there is provided pharmaceutical composition comprising one or more compounds of the present disclosure, or a pharmaceutically acceptable salt thereof, and at least one pharmaceutical acceptable excipient.

As used herein, the term “pharmaceutical composition” refers to a formulation containing the molecules or compounds of the present disclosure in a form suitable for administration to a subject.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used herein includes both one and more than one such excipient. The term “pharmaceutically acceptable excipient” also encompasses “pharmaceutically acceptable carrier” and “pharmaceutically acceptable diluent”.

The particular excipient used will depend upon the means and purpose for which the compounds of the present disclosure is being applied. Solvents are generally selected based on solvents recognized by persons skilled in the art as safe to be administered to a mammal including humans. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof.

In some embodiments, suitable excipients may include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

In some embodiments, suitable excipients may include one or more stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present disclosure or pharmaceutical composition thereof) or aid in the manufacturing of the pharmaceutical product (i.e., medicament). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). A “liposome” is a small vesicle composed of various types of lipids, phospholipids and/or surfactant which is useful for delivery of a drug (such as the compounds disclosed herein and, optionally, a chemotherapeutic agent) to a mammal including humans. The components of the liposome are commonly arranged in a bilayer formation, similar to the lipid arrangement of biological membranes.

The pharmaceutical compositions provided herein can be in any form that allows for the composition to be administered to a subject, including, but not limited to a human, and formulated to be compatible with an intended route of administration.

A variety of routes are contemplated for the pharmaceutical compositions provided herein, and accordingly the pharmaceutical composition provided herein may be supplied in bulk or in unit dosage form depending on the intended administration route. For example, for oral, buccal, and sublingual administration, powders, suspensions, granules, tablets, pills, capsules, gelcaps, and caplets may be acceptable as solid dosage forms, and emulsions, syrups, elixirs, suspensions, and solutions may be acceptable as liquid dosage forms. For injection administration, emulsions and suspensions may be acceptable as liquid dosage forms, and a powder suitable for reconstitution with an appropriate solution as solid dosage forms. For inhalation administration, solutions, sprays, dry powders, and aerosols may be acceptable dosage form. For topical (including buccal and sublingual) or transdermal administration, powders, sprays, ointments, pastes, creams, lotions, gels, solutions, and patches may be acceptable dosage form. For vaginal administration, pessaries, tampons, creams, gels, pastes, foams and spray may be acceptable dosage form.

In some embodiments, the pharmaceutical compositions of the present disclosure may be in a form of formulation for oral administration.

In certain embodiments, the pharmaceutical compositions of the present disclosure may be in the form of tablet formulations. Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

In certain embodiments, the pharmaceutical compositions of the present disclosure may be in a form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

In certain embodiments, the pharmaceutical compositions of the present disclosure may be in the form of aqueous suspensions, which generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

In certain embodiments, the pharmaceutical compositions of the present disclosure may be in the form of oily suspensions, which generally contain suspended active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

In certain embodiments, the pharmaceutical compositions of the present disclosure may be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

In certain embodiments, the pharmaceutical compositions provided herein may be in the form of syrups and elixirs, which may contain sweetening agents such as glycerol, propylene glycol, sorbitol, aspartame or sucrose, a demulcent, a preservative, a flavoring and/or coloring agent.

In some embodiments, the pharmaceutical compositions of the present disclosure may be in a form of formulation for injection administration.

In certain embodiments, the pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

In some embodiments, the pharmaceutical compositions of the present disclosure may be in a form of formulation for inhalation administration.

In certain embodiments, the pharmaceutical compositions of the present disclosure may be in the form of aqueous and nonaqueous (e.g., in a fluorocarbon propellant) aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols.

In some embodiments, the pharmaceutical compositions of the present disclosure may be in a form of formulation for topical or transdermal administration.

In certain embodiments, the pharmaceutical compositions provided herein may be in the form of creams, ointments, gels and aqueous or oily solutions or suspensions, which may generally be obtained by formulating an active ingredient with a conventional, topically acceptable excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In certain embodiments, the pharmaceutical compositions provided herein may be formulated in the form of transdermal skin patches that are well known to those of ordinary skill in the art.

Besides those representative dosage forms described above, pharmaceutically acceptable excipients and carriers are generally known to those skilled in the art and are thus included in the present disclosure. Such excipients and carriers are described, for example, in “Remingtons Pharmaceutical Sciences” Mack Pub. Co., New Jersey (1991), in “Remington: The Science and Practice of Pharmacy”, Ed. University of the Sciences in Philadelphia, 21^{st} Edition, LWW (2005), which are incorporated herein by reference.

In some embodiments, the pharmaceutical compositions of the present disclosure can be formulated as a single dosage form. The amount of the compounds provided herein in the single dosage form will vary depending on the subject treated and particular mode of administration.

In some embodiments, the pharmaceutical compositions of the present disclosure can be formulated so that a dosage of between 0.001-1000 mg/kg body weight/day.

In some embodiments, the pharmaceutical compositions of the present disclosure can be formulated as short-acting, fast-releasing, long-acting, and sustained-releasing. Accordingly, the pharmaceutical formulations of the present disclosure may also be formulated for controlled release or for slow release.

In a further aspect, there is also provided veterinary compositions comprising one or more molecules or compounds of the present disclosure or pharmaceutically acceptable salts thereof and a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

The pharmaceutical compositions or veterinary compositions may be packaged in a variety of ways depending upon the method used for administering the drug. For example, an article for distribution can include a container having deposited therein the compositions in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings. The compositions may also be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described.

In a further aspect, the present disclosure provides pharmaceutical compositions comprise one or more compounds of the present disclosure, or a pharmaceutically acceptable salt thereof, as a first active ingredient, and a second active ingredient. The second active ingredient can be any suitable active ingredient for the method of treatment, including, without limitation, an anti-cancer therapy, or in particular, an anti-prostate cancer drug.

Methods of Treating a Disease or Condition

In one aspect, the present disclosure provides a method of treating an androgen receptor (AR)-associated disease or condition in a subject, comprising administering to the subject a therapeutically effective amount of an AR condensate modulator, wherein the AR condensate modulator modulates an AR condensate comprising at least AR.

In certain embodiments, the AR condensate modulator comprises a compound selected from the group consisting of. Compound 60, Compound 61, Compound 62, Compound 6, or Compound 2, or a pharmaceutically acceptable salt, solvate, prodrug, enantiomer, diastereomer, tautomer, isotopic substitution, polymorph or metabolite thereof.

In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art. The therapeutically effective amount is varied according to the particular treatment involved for a subject and depend upon various factors known in the art, such as the subject's body weight, size, and health; the nature and extent of the condition; the rate of administration; the therapeutic or combination of therapeutics selected for administration; and the discretion of the prescribing physician. Therapeutically effective amounts for a given situation can be determined by routine experimentation that is within the skill and judgment of the clinician. For example, the initial administration dosage may be higher than subsequent administration dosages. For another example, the administration dosage may vary over the course of treatment depending on the reaction of the subject.

In some embodiments, wherein the AR-associated disease or condition is characterized in having an abnormal level of AR activity.

As used herein, the term “abnormal level of AR activity” refers to AR activity the level of which is substantially lower or higher than the normal or baseline level of AR activity. Normal level of AR activity can be a level of AR activity in the healthy cell or tissue sample. A baseline level of AR activity can be an average level of the AR activity in the general cancer patient population, or in a general patient population of AR dependent cancers such as AR dependent prostate cancer.

In some embodiments, the AR-associated disease or condition is characterized in having an elevated level of AR activity.

In some embodiments, the AR-associated disease or condition is characterized having one or more genetic aberrations that results in elevated AR activity, constitutive AR activation, or resistance to castration or androgen deprivation therapy.

In some embodiments, the one or more genetic aberrations comprise amplification of AR gene, mutations in AR gene, aberrant splicing of AR gene, rearrangement in AR gene, polymorphism in AR gene, or any combination thereof.

In some embodiments, the AR-associated disease or condition is characterized having an AR variant, optionally, the AR variant lacks all or part of the ligand binding domain (LBD).

In some embodiments, the AR-associated disease or condition is prostate cancer, breast cancer, glioblastoma, melanoma, bladder cancer, renal cell carcinoma, pancreatic cancer, hepatocellular carcinoma, ovarian cancer, endometrial cancer, mantle cell lymphoma, salivary gland cancer, hair loss, acne, hirsutism, ovarian cysts, polycystic ovary disease, precocious puberty, spinal and bulbar muscular atrophy, or age-related macular degeneration.

In some embodiments, the AR-associated disease or condition is an AR-expressing cancer.

In some embodiments, the AR-expressing cancer is prostate cancer, breast cancer, glioblastoma, melanoma, bladder cancer, renal cell carcinoma, pancreatic cancer, hepatocellular carcinoma, ovarian cancer, endometrial cancer, mantle cell lymphoma, salivary gland cancer.

In some embodiments, the AR-expressing cancer is metastatic.

In some embodiments, the AR-expressing cancer is prostate cancer. In some embodiments, the prostate cancer is primary or localized prostate cancer, locally advanced prostate cancer, recurrent prostate cancer, advanced prostate cancer, metastatic prostate cancer, metastatic castration-resistant prostate cancer, and hormone-sensitive prostate cancer.

In some embodiments, the prostate cancer is resistant to castration or androgen deprivation therapy (ADT). By “resistant” it is meant that the disease has no or reduced responsiveness or sensitivity to an ADT. Reduced responsiveness can be indicated by, for example, requirement of an increased dose to achieve a given efficacy. In certain embodiments, the prostate cancer can be non-responsive to an ADT. For example, the cancer cells or tumor size increases despite of the treatment with the an ADT, or the disease showed regression or recurrence back to its former state, for example, return of previous symptoms following partial recovery. The resistance to an ADT can be de novo or acquired.

In some embodiment, the prostate cancer is castration-resistant. Castration-resistant prostate cancer (CRPC) is an advanced prostate cancer that shows disease progression despite of androgen deprivation therapy. In some embodiments, CRPC involves elevated expression level or activity of AR. In some embodiments, CRPC involves mutated AR having androgen-independent AR activation. In some embodiments, CRPC involves intratumoral and alternative androgen production.

In some embodiment, the AR condensate modulator is administered in combination with a second active ingredient or therapy. The second active ingredient or therapy can be any suitable active ingredient or therapy for the method of treatment, including, without limitation, an anti-cancer therapy, or in particular, an anti-prostate cancer drug.

Examples of anti-cancer therapy include, without limitation, a chemotherapeutic agent, radiation therapy, an immunotherapy agent, anti-angiogenesis agent, a cellular therapy agent, a gene therapy agent, a hormonal therapy agent, cytokines, palliative care, surgery for the treatment of cancer, one or more anti-emetics, treatments for complications arising from chemotherapy.

The second active ingredient can be an anti-prostate cancer drug. Examples of the anti-prostate cancer drug comprises an androgen axis inhibitor; an androgen synthesis inhibitor; a poly ADP-ribose polymerase (PARP) inhibitor; or a combination thereof.

In certain embodiments, the androgen axis inhibitor is selected from the group consisting of Luteinizing hormone-releasing hormone (LHRH) agonists, LHRH antagonists and androgen receptor antagonist.

In certain embodiments, the androgen axis inhibitor is degarelix, bicalutamide, flutamide, nilutamide, apalutamide, darolutamide, enzalutamide, or abiraterone. In certain embodiments, the androgen synthesis inhibitor is abiraterone acetate or ketoconazole. In certain embodiments, the PARP inhibitor is olaparib, or rucaparib.

In certain embodiments, the anti-prostate cancer drug is selected from the group consisting of Abiraterone Acetate, Apalutamide, Bicalutamide, Cabazitaxel, Casodex (Bicalutamide), Darolutamide, Degarelix, Docetaxel, Eligard (Leuprolide Acetate), Enzalutamide, Erleada (Apalutamide), Firmagon (Degarelix), Flutamide, Goserelin Acetate, Jevtana (Cabazitaxel), Leuprolide Acetate, Lupron (Leuprolide Acetate), Lupron Depot (Leuprolide Acetate), Lynparza (Olaparib), Mitoxantrone Hydrochloride, Nilandron (Nilutamide), Nilutamide, Nubeqa (Darolutamide), Olaparib, Provenge (Sipuleucel-T), Radium 223 Dichloride, Rubraca (Rucaparib Camsylate), Rucaparib Camsylate, Sipuleucel-T, Taxotere (Docetaxel), Xofigo (Radium 223 Dichloride), Xtandi (Enzalutamide), Zoladex (Goserelin Acetate) and Zytiga (Abiraterone Acetate).

Methods of Modulation of Transcription

In another aspect, the present disclosure provides a method of modulating transcription of one or more AR-regulated genes in a cell or a subject, comprising modulating a transcriptional AR condensate comprising at least AR.

Regulating transcription of a gene can, for example, include one or more of the following events: increasing or decreasing the rate or frequency of gene transcription, increasing or reducing inhibition of gene transcription, and increasing or decreasing mRNA transcription initiation, mRNA elongation, or mRNA splicing activity.

In some embodiment, the transcription of one or more genes is modulated by modulating the transcriptional AR condensate.

In some embodiments, the AR is a wild type AR or an AR variant.

In some embodiments, the method comprises modulating the transcriptional AR condensate by an AR condensate modulator.

In some embodiments, the AR condensate modulator modulates formation, stability, or activity of the AR condensate.

In some embodiments, the AR condensate modulator is an AR condensate inhibitor.

In some embodiments, the AR-regulated gene is an AR target gene or an AR variant target gene.

In some embodiments, the AR target gene comprises FKBP5, KLK2, PSA, TMPRSS2, NKX3.1, or any combination thereof.

In some embodiments, the AR variant target gene comprises BUB1B, CCNA2, UBE2C, KIF15, CDC20, or any combination thereof.

In some embodiments, the AR condensate inhibitor binds to the intrinsic disorder domain (IDD) of AR.

In some embodiments, the AR condensate modulator comprises a peptide, nucleic acid, or small molecule.

In some embodiments, the cell or subject is characterized in having an abnormal level of AR activity, or an elevated level of AR activity.

In some embodiments, the cell or subject is characterized in having one or more genetic aberrations that results in elevated AR activity, constitutive AR activation, or resistance to castration or androgen deprivation therapy.

In some embodiments, the one or more genetic aberrations comprise amplification of AR gene, mutations in AR gene, aberrant splicing of AR gene, rearrangement in AR gene, polymorphism in AR gene, or any combination thereof.

In some embodiments, the cell or subject is characterized in having an AR variant, optionally, the AR variant lacks all or part of the ligand binding domain (LBD).

In Vitro Screening System

In another aspect, the present disclosure provides an in vitro screening system comprising AR or an intrinsic disorder domain containing fragment thereof, wherein the AR or the fragment is attached to a detectable label and is capable of forming an AR condensate.

In some embodiments, the AR is a wild type AR or an AR variant provided herein.

In some embodiment, in vitro screening system is based on naturally occurring AR condensates in a cell. The cell can be a transgenic cell or otherwise manipulated cell. In some embodiments, in vitro screening system is based on in vitro AR condensates. In some embodiments, the in vitro AR condensate comprises components mimicking a condensate found in a cell.

In some embodiments, the in vitro screening system comprises a cell or a nucleus of a cell.

In some embodiments, the in vitro screening system comprises a cell lysate or a nuclear lysate. In some embodiments, an in vitro AR condensate is isolated from a cell. Any suitable means of isolation of a condensate from a cell or composition is encompassed herein (e.g., chemically or immunologically precipitated). In some embodiments, a condensate is isolated by centrifugation (e.g., at about 5,000×g, 10,000×g, 15,000×g for about 5-15 minutes; about 10,000×g for about 10 min). A condensate may be isolated from a cell by lysis of the nucleus of a cell with a homogenizer (i.e., Dounce homogenizer) under suitable buffer conditions, followed by centrifugation and/or filtration to separate the condensate.

In some embodiments, in vitro AR condensates are synthetic condensates with one or more condensate components in a solution.

In some embodiments, the detectable label comprises a fluorophore, a radioisotope, a colorimetric substrate, or an antigenic epitope. The term “fluorophore” as used herein includes any fluorescent molecules or moieties, including fluorescent proteins, peptides, chemical compounds, and the like. The term “detectable label” as used herein includes, but is not limited to, fluorophores, radioisotopes, colorimetric substrates, or enzymes; heterologous epitopes for which specific antibodies are commercially available, e.g., FLAG-tag; heterologous amino acid sequences that are ligands for commercially available binding proteins, e.g., Strep-label, biotin; fluorescence quenchers typically used in conjunction with a fluorescent tag on the other polypeptide; and complementary bioluminescent or fluorescent polypeptide fragments.

A label that is a detectable label or a complementary bioluminescent or fluorescent polypeptide fragment may be measured directly (e.g., by measuring fluorescence or radioactivity of, or incubating with an appropriate substrate or enzyme to produce a spectrophotometrically detectable color change for the associated polypeptides as compared to the unassociated polypeptides). A label that is a heterologous epitope or ligand is typically detected with an additional agent that binds thereto, e.g., an antibody or binding protein, wherein the agent is associated with a detectable label. In some embodiments, AR or a condensate component (e.g., the first or second component as described herein) comprises a detectable label.

In some embodiments, the in vitro condensate comprises a plurality of detectable labels as described herein. In some embodiments, different detectable labels are attached to AR and different components of AR condensate (e.g., AR or a fragment thereof labeled with one fluorescent label and RNA polymerase Pol II labeled with a different fluorescent label). In some embodiments, one or more components of the condensate have a quencher.

In some embodiments, the in vitro screening system further comprises a DNA/RNA sequence, histone, cofactor, mediator, RNA polymerase, or any combination thereof.

Synthetic Condensate

In another aspect, the present disclosure provides a synthetic AR condensate comprising at least AR or a fragment thereof comprising IDD.

As used herein, a “synthetic” condensate refers to a non-naturally occurring condensate comprising condensate components. In some embodiment, the synthetic AR condensate is a synthetic transcriptional AR condensate. In some embodiments, the synthetic AR condensate simulates a transcriptional AR condensate found in a cell.

The synthetic AR condensates may comprise any of the components described herein. In some embodiments, the AR is a wild type AR or an AR variant provided herein.

In some embodiments, the condensate further comprises a DNA/RNA sequence, histone, cofactor, mediator, RNA polymerase, or any combination thereof.

In some embodiments, the fragment of AR can form or incorporate into a condensate under relevant physiological conditions (e.g., conditions the same as or approximating conditions in a cell) or relevant experimental conditions (e.g., suitable conditions for the formation of a condensate in vitro).

In some embodiments, AR or a fragment thereof further comprises a detectable label as described herein. In some aspects, the detectable label is a fluorescent label.

Some aspects of the disclosure provide methods of making synthetic transcriptional AR condensates. In some embodiments the method comprises combining AR or a fragment thereof comprising IDD, optionally with two or more condensate components, in vitro under conditions suitable for formation of transcriptional AR condensates. The conditions can include appropriate concentrations of components, salt concentration, pH, etc. In some embodiments, the conditions include a salt concentration (e.g., NaCl) of about 25 nM, 40 mM, 50 mM, 125 mM, 200 mM, 350 mM, or 425 mM; or in the range of about 10-250 mM, 25-150 mM, or 40-100 mM. In some embodiments, the conditions include a pH of about 7-8, 7.2-7.8, 7.3-7.7, 7.4-7.6, or about 7.5.

Modified Host Cell

In another aspect, the present disclosure provides a modified host cell expressing AR or an intrinsic disorder domain containing fragment thereof, wherein the AR or the fragment is attached to a detectable label and is capable of forming an AR condensate.

“Host cell” as used herein refers to a eukaryotic cell to which an expression vector encoding an exogenous protein or peptide is introduced, as well any progeny or potential progeny of such a cell. Because certain modifications may occur in succeeding generations due to either mutation or environmental influences, such progeny may not, in fact, be identical to the parent cell, but are still included within the scope of the term as used herein.

In some embodiments, the AR is a wild type AR or an AR variant.

In some embodiments, the detectable label comprises a fluorophore, a radioisotope, a colorimetric substrate, or an antigenic epitope.

In some embodiments, the modified host cell is suitable for detection of formation of the AR condensate.

In some embodiments, the host cell is a tumor cell, optionally a prostate cancer cell.

The modified host cell as disclosed herein can be used to produce the in vitro screening system provided herein. In one embodiment, the host cell is cultured (into which a recombinant expression vector encoding an AR or a fragment fused to a detectable tag has been introduced) in a suitable medium until AR or a fragment thereof is produced, and then a composition comprising an AR condensate is isolated from the cell.

The modified host cells of the invention can also be used to produce nonhuman transgenic animals. The nonhuman transgenic animals can be used in screening assays designed to identify agents which are capable of modulating AR condensate and ameliorating detrimental symptoms of cancer.

Method of Screening

In another aspect, the present disclosure provides a method of screening for an agent that modulates an AR condensate comprising at least AR, comprising:
a) providing the AR condensate and assessing one or more physical properties or one or more biological effects of the AR condensate,
b) contacting the AR condensate with a test agent, and
c) assessing whether the test agent causes a change in the one or more physical properties or one or more biological effects of the AR condensate.

In some embodiments, the test agent is identified as modulating the condensate if it causes a change in the one or more physical properties or one or more biological effects of the AR condensate. In some embodiments, the physical properties comprises formation, composition, stability, and/or activity of the AR condensate.

In some embodiment, the physical properties of an AR condensate is measured. Physical properties can include, without limitation, composition, stability, size, concentration, permeability, morphology and viscosity. Any suitable method known in the art may be used to measure the one or more physical properties.

In some embodiments, the condensate has a detectable label and the detectable label is used to determine if contact with the test agent causes any changes in the one or more physical properties or one or more biological effects of the AR condensate.

In some embodiments, the test agent is assessed to determine whether one or more of the following physical properties of an AR condensate is change upon the contact (i) number of AR condensates; (ii) size of AR condensates; (iii) location of AR condensates; (iv) distribution of AR condensates. (v) surface area of AR condensates; (vi) composition of AR condensates; (vii) liquidity of AR condensates; (viii) solidification of AR condensates; and (ix) dissolution of AR condensate

These physical properties can correlate with the condensate's ability to activate a reporter gene.

In some embodiments, the AR condensate is contained in an in vitro screening system provided herein, in a synthetic AR condensate provided herein, or in a modified host cell provided herein.

In some embodiments, the AR condensate is an isolated synthetic condensate, or is in the form of an isolated cellular composition comprising the AR condensate.

In some embodiments, the method of screening is performed in a cell-free system, comprising provide an isolated cellular composition comprising a AR condensate, contacting the composition with a test agent, and determining if contact with the test agent causes a change in the one or more physical properties or one or more biological effects of the AR condensate. In some embodiments, the isolated cellular composition comprising a nucleus comprising an AR condensate.

The type of cell having a condensate or from which a composition having an AR condensate is isolated is not limited and may be any cell type disclosed herein. In some embodiments, the cell is affected by a disease (e.g., a cancer cell). In some embodiments, the cell having a condensate is a primary cell, a member of a cell line, cell isolated from a subject suffering from a disease, or a cell derived from a cell isolated from a subject suffering from a disease (e.g., a progenitor of an induced pluripotent cell isolated from a subject suffering from a disease).

In some embodiments, the AR condensate is a synthetic condensate (also see description below). A synthetic condensate can appear as a liquid droplets in vitro composed of AR and one or more components (e.g., the first and second components as described herein). Such droplets may further comprise RNA, DNA and/or histones. Such liquid droplets are in vitro condensates and can correspond to and/or serve as models of condensates that exist in vivo.

In some embodiment, the physical properties of an AR condensate is measured. Physical properties can include, without limitation, composition, stability, size, concentration, permeability, morphology and viscosity. Any suitable method known in the art may be used to measure the one or more physical properties. These physical properties can correlate with the condensate's ability to activate a reporter gene in a cell.

The condensate may be a naturally occurring condensate. In some embodiments, the AR condensate is inside a cell or inside nucleus. In other embodiments, the condensate may occur in a transgenic cell or an otherwise manipulated cell. In some embodiments, the method of screening is performed in a cell-based system, comprising providing a cell having an AR condensate, contacting the cell with a test agent, and determining if contact with the test agent causes a change in the one or more physical properties or one or more biological effects of the AR condensate.

In some embodiments, the AR condensate is a transcriptional AR condensate. In some embodiments, the transcriptional condensate further comprises a RNA polymerase.

In another aspect, the present disclosure provides a method of identifying an agent that modulates formation of an AR condensate comprising at least AR, comprising:
a) providing components capable of forming the AR condensate;
b) contacting the components with a test agent under the condition suitable for formation of the AR condensate, and
c) assessing whether presence of the test agent affects formation of the AR condensate or one or more biological effects of the AR condensate.

In some embodiments, the test agent is identified as modulating the formation of the condensate if it affects formation of the AR condensate or affects the one or more biological effects of the AR condensate.

In some embodiments, the components capable of forming the AR condensate are contained in an in vitro screening system provided herein, in a synthetic AR condensate provided herein, or in a modified host cell provided herein.

In some embodiments, the cell is a genetically engineered to express the detectable label.

In some embodiments, the AR condensate is a transcriptional condensate.

In some embodiments, the one or more biological effects of the transcriptional condensate is assessed based on expression of an AR-regulated gene or cell proliferation.

In some embodiments, the AR-regulated gene is an AR target gene, an AR variant target gene, or a reporter gene.

In some embodiments, the test agent has been identified as capable of binding to IDD of AR.

Any suitable method of detecting changes of the condensate upon the contacting of the test agent may be used, including methods known in the art and taught herein. In some embodiments, the step of determining if contact with the test agent causes changes to properties of a condensate is performed using microscopy. In some embodiments, the microscopy is deconvolution microscopy, structured illumination microscopy, or interference microscopy. In some embodiments, the step of determining if contact with the test agent causes changes to properties of a condensate is performed using DNA-FISH, RNA-FISH, or a combination thereof.

In some embodiments, one or more biological effects of the transcriptional AR condensate is assessed based on expression of a target gene in a condensate-dependent manner. In some embodiments, the target gene is a reporter gene. Such reporter gene can be operatively linked to a binding site for AR. In some embodiments, the reporter gene encode a fluorescent or luminescent protein that are detectable.

In some embodiments, the test agent is identified as modulating the formation of the condensate if it affects formation of the condensate or affects the one or more biological effects of the AR condensate.

For example, one can provide AR, and optionally other components, combine them in a vessel, and observe what happens in terms of condensate formation and/or measure the properties (e.g., increases or decreases in stability, composition, activity, morphology) of resulting condensates. In some embodiments, the provided composition will form a condensate and the test agent will be screened for modulating formation (e.g., increasing or decreasing condensate formation or the rate of condensate formation).

In some embodiments, a method of screening an agent that modulates a AR condensate formation comprises providing a cell, an isolated cellular composition and/or an in vitro transcription assay expressing a reporter gene under the control of a AR condensate, contacting the cell or assay with a test agent, and assessing expression of the reporter gene.

In some embodiments, the method may be performed to identify an agent that interacts with AR and drives AR into a transcriptional condensate. In some embodiments, the method may be performed to identify an agent that interacts with AR and prevents integration of AR into a condensate. In some embodiments, the method may be performed to identify an agent that force integration of a component into an AR condensate or prevent a component from entering an AR condensate. In some embodiments, an agent identified by the methods disclosed herein of modulating an AR condensate or the formation of an AR condensate is further tested for its ability to modulate one or more features of a disease. The disease is not limited and may be any disease disclosed herein. For example, if the agent inhibits the expression of a reporter gene or the formation of an AR condensate, could test the ability of the agent to inhibit proliferation of cancer cells that has an elevated expression of AR relative to a reference.

In some embodiments, an agent identified as modulating one or more physical properties or formation of a condensate (e.g., formation, stability, or morphology) or functional properties of a condensate (e.g. modulation of transcription) by the methods disclosed herein may be administered to a subject, e.g., a non-human animal that serves as a model for a disease, or a subject in need of treatment for the disease.

In some embodiments, a high throughput screen (HTS) is performed. A high throughput screen can utilize either cell-free or cell-based assays (e.g., a condensate containing cell, a synthetic condensate, an isolated cellular composition). High throughput screens often involve testing large numbers of test agents with high efficiency, e.g., in parallel. For example, tens or hundreds of or thousands of compounds can be routinely screened in short periods of time, e.g., hours to days. Often such screening is performed in multiwell plates containing, at least 96 wells or other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarron R & Hertzberg RP. Design and implementation of high-throughput screening assays. Methods Mol Biol., 565:1-32, 2009 and/or An WF & Tolliday NJ., Introduction: cell-based assays for high-throughput screening. Methods Mol Biol. 486:1-12, 2009, and/or references in either of these.

In some embodiments, an analog of an agent identified as modulating one or more physical properties or formation of a condensate (e.g., formation, stability, function, or morphology) or functional properties of a condensate (e.g. modulation of transcription) by the methods disclosed herein may be generated. An “analog” of a first agent refers to a second agent that is structurally and/or functionally similar to the first agent. An analog of an agent may have substantially similar physical, chemical, biological, and/or pharmacological propert(ies) as the agent or may differ in at least one physical, chemical, biological, or pharmacological property. In some embodiments at least one such property differs in a manner that renders the analog more suitable for a purpose of interest, e.g., for modulating a condensate. Methods of generating analogs are known in the art and include methods described herein. In some embodiments, generated analogs can be tested for a property of interest, such as increased stability (e.g., in an aqueous medium, in human blood, in the GI tract, etc.), increased bioavailability, increased half-life upon administration to a subject, increased cell uptake, increased activity to modulate a condensate property including physical properties or formation of a condensate (e.g., formation, stability, function, or morphology) or functional properties of a condensate (e.g. modulation of transcription), increased specificity for a condensate.

### EXAMPLES

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Example 1

### Synthesis of Compound 2, compound 6, Compound 60, Compound 61, Compound 62, Compound AA

### Synthesis of Intermediate A1: 3-chloro-2-(2-chloroethoxy)-5-(2-(4-hydroxyphenyl)propan-2-yl)benzonitrile

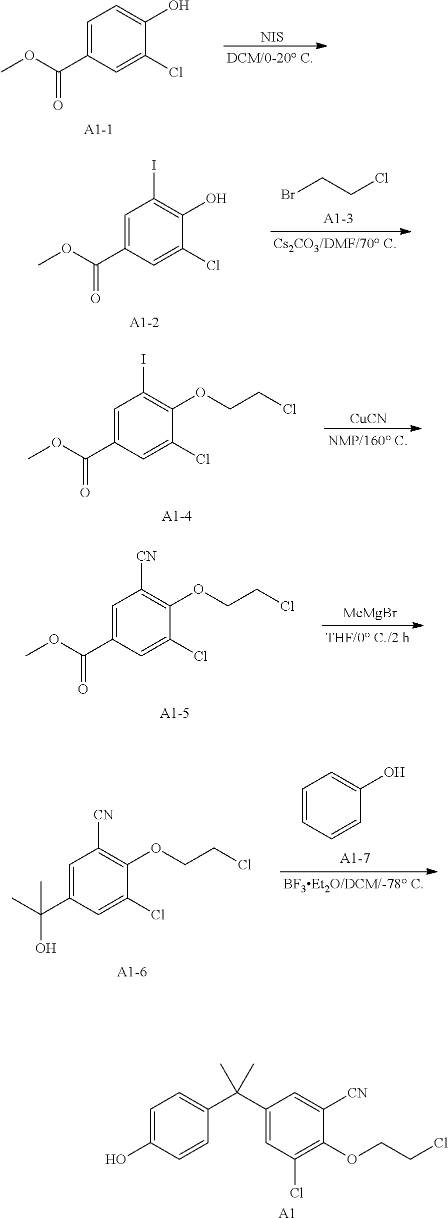

Step 1: To a solution of A1-1 (80 g, 429 mmol) in Dichloromethane (1000 mL) was added NIS (116 g, 515 mmol) at 0° C. Then the solution was stirred at room temperature for 16 hours. The solution was poured onto water and extracted with Dichloromethane (1000 ml*2). The organic layer was combined, washed with brine, dried with Na₂SO₄ and evaporated to dryness. The residue was re-crystalized with CH₃CN to yield product as white solid. A1-2 (117 g, yield 87.3%) LC-MS [M−1]⁻=310.7

Step 2: To a solution of A1-2 (100 g, 320 mmol), A1-3 (91 g, 640 mmol) and DMF (1200 mL) was added Cs₂CO₃ (208 g, 640 mmol). The mixture was stirred at 70° C. for 16 hours. The mixture was poured onto water and extracted with ethyl acetate (1000 ml*3). The organic layer was combined, washed with brine, dried with Na₂SO₄, filtered and evaporated to dryness. The residue was purified with silica column (PE/ethyl acetate=10/1) to afford A1-4 (103.4 g, 86.1%) as colorless oil. LC-MS[M+1]⁺=374.7

Step 3: To a solution of A1-4 (53 g, 141.3 mmol) in NMP(600 mL) was added CuCN(19 g, 200 mmol). The mixture was stirred at 160° C. for 3 hours under N₂ atmosphere. The mixture was quenched with ammonia water (2000 mL, 23%) and then extracted with ethyl acetate (1000 mL*3). The organic layer was combined, washed with brine, dried with Na₂SO₄, filtered and evaporated to dryness. The residue was purified with silica column (PE/ethyl acetate=10/1) to afford A1-5 (21 g, 54.3%) as white solid.

LC-MS [M+1]⁺=274.0

Step 4: To a solution of A1-5 (21 g, 76.6 mmol) in THF(100 mL) was added MeMgBr solution(613 mL, 1M solution). The mixture was stirred at room temperature for 2 hours under N₂ atmosphere. The mixture was poured onto saturated ammonia chloride solution and then extracted with ethyl acetate (300 mL*3). The organic layer was combined, washed with brine, dried with Na₂SO₄, filtered and evaporated to dryness. The residue was purified with silica column (PE/ethyl acetate=5/1) to afford A1-6 (17 g, 80.9%) as yellow oil. LC-MS [M+17]⁺=290.9

Step 5: To a solution of A1-6 (19.2 g, 310 mmol) and A1-7 (19.2 g, 310 mmol) in DCM(200 mL) was added BF₃·Et₂O solution(34 mL, 47%) at −78° C. under N₂ atmosphere. The mixture was stirred at room temperature for 2 hours. The mixture was poured onto water(200 mL) and then extracted with ethyl Dichloromethane (100 mL*3). The organic layer was combined, washed with brine, dried with Na₂SO₄, filtered and evaporated to dryness. The residue was purified with silica column (PE/ethyl acetate=5/1) to afford A1 (18 g, 83%) as yellow oil.

¹H NMR (400 MHz, Chloroform-d) δ 7.44 (d, J=2.4 Hz, 1H), 7.33 (dd, J=4.9, 2.4 Hz, 1H), 7.06-7.02 (m, 2H), 6.80-6.76 (m, 2H), 4.41 (t, J=6.2 Hz, 2H), 3.87 (t, J=6.2 Hz, 2H), 1.63 (s, 6H).

LC-MS [M−1]⁻=347.9

### Synthesis of Intermediate A2: 3-chloro-2-(2-chloroethoxy)-5-(2-(4-ethynylphenyl) propan-2-yl) benzonitrile

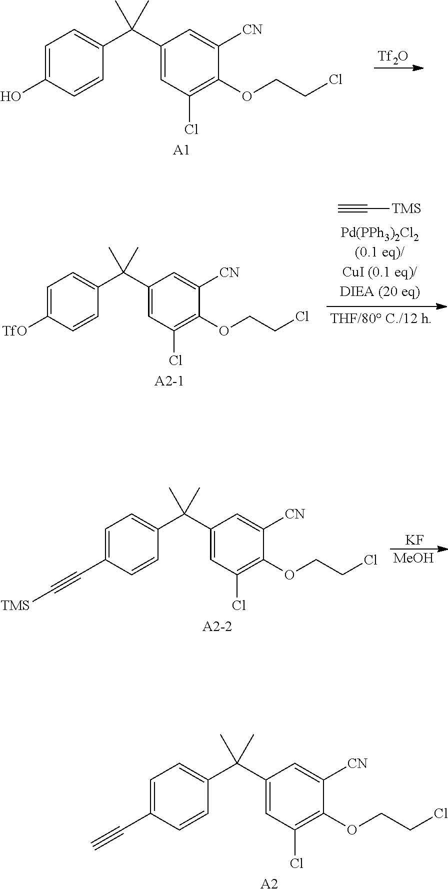

Step 1: To a solution of A1 (5.8 g, 16 mmol), Et₃N (4.87 g, 48 mmol) in DCM(100 mL) was added Tf₂O(6.78 g, 24 mmol). The mixture was stirred at 25° C. for 2 hours under N₂ atmosphere. The mixture was quenched with water (100 mL) and then extracted with DCM (200 mL*3). The organic layer was combined, washed with brine, dried with Na₂SO₄, filtered and evaporated to dryness. The residue was purified with silica column (PE/ethyl acetate=30/1) to afford A2-1 (6 g, 78%) as orange oil.

Step 2: To a solution of A2-1 (6 g, 12.5 mmol), ethynyltrimethylsilane(12 g, 122.2 mmol), Pd(PPh₃)₂Cl₂ (1.6 g, 2.3 mmol), Et₃N (23.31 g, 230.4 mmol) in CH₃CN(100 mL) was added CuI(1.3 g, 6.8 mmol). The mixture was stirred at 80° C. for 16 hours under N₂ atmosphere. The mixture was quenched with water (100 mL) and then extracted with DCM (300 mL*3). The organic layer was combined, washed with brine, dried with Na₂SO₄, filtered and evaporated to dryness. The residue was purified with silica column to afford A2-2 (4.2 g, 69%) as orange oil.

Step 3: To a solution of A2-2 (4.2 g, 9.79 mmol) in MeOH (65 mL) was added KF(4.2 g, 72.3 mmol). The mixture was stirred at room temperature for 12 hours under N₂ atmosphere. The mixture was quenched with water (50 mL) and then extracted with DCM (300 mL*3) The organic layer was combined, washed with brine, dried with Na₂SO₄, filtered and evaporated to dryness. The residue was purified with silica column to afford A2 (2.8 g, 80%) as orange oil.

¹H NMR (400 MHz, Chloroform-d) δ 7.48-7.42 (m, 2H), 7.41 (d, J=2.4 Hz, 1H), 7.33 (d, J=2.4 Hz, 1H), 7.16-7.11 (m, 2H), 4.42 (t, J=6.2 Hz, 2H), 3.88 (t, J=6.2 Hz, 2H), 3.07 (s, 1H), 1.65 (s, 6H).

### The synthesis of compound A3: 3-chloro-5-(2-(4-ethynylphenyl)propan-2-yl)-2-methoxybenzonitrile

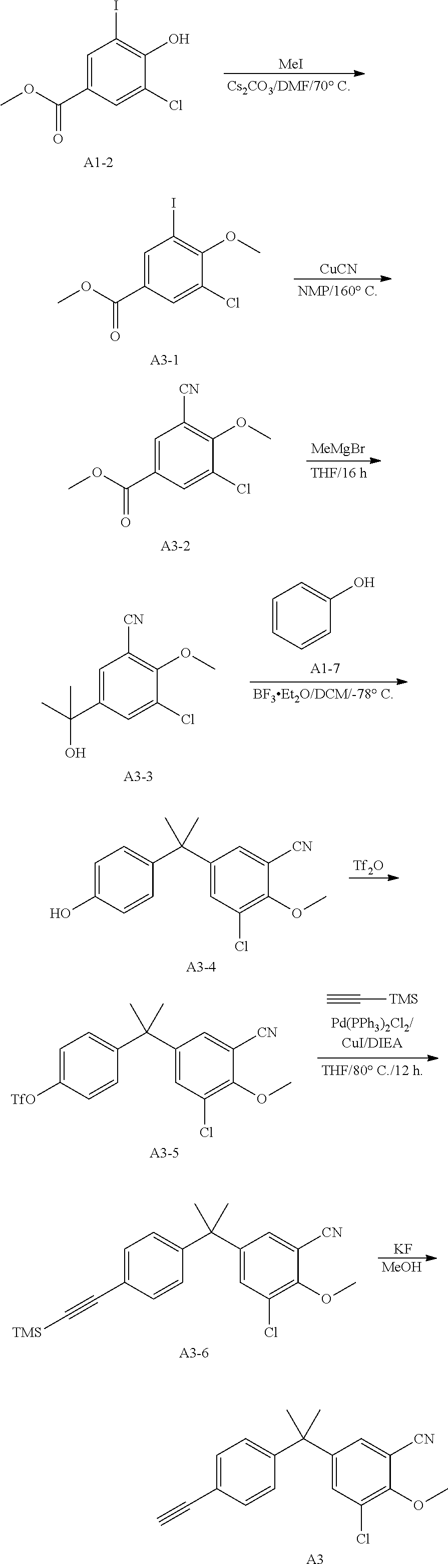

Following the same procedure of compound A1, using iodomethane to replace the A1-3, to prepare the compound A3-4 through 4 steps.

LC-MS[M+18]⁺=319.1

Following the same procedure of compound A2, using A3-4 to replace the A1, to prepare the 3-chloro-5-(2-(4-ethynylphenyl)propan-2-yl)-2-methoxybenzonitrile (compound A3) through 3 steps.

¹H NMR (400 MHz, Chloroform-d) δ 7.43 (d, J=7.0 Hz, 2H), 7.37 (d, J=2.2 Hz, 1H), 7.31 (d, J=2.2 Hz, 1H), 7.15-7.11 (m, 2H), 4.04 (s, 3H), 3.06 (s, 1H), 1.64 (s, 6H).

LC-MS[M+1]⁺=310.1

### Synthesis of Intermediate B1: 1-iminotetrahydro-1H-1l6-thiophene 1-oxide

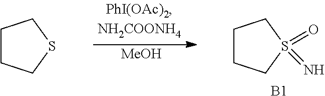

Step 1: To a solution of tetrahydrothiophene (0.2 mL, 2.27 mmol) NH₂COONH₄ (177.1 mg, 2.27 mmol) in MeOH (5 mL) was added PhI(OAc)₂ (1826.6 mg, 5.67 mmol). The mixture was stirred at room temperature for 1 hour. The mixture was quenched with water (10 mL) and then extracted with DCM (50 mL*3). The organic layer was combined, washed with brine, dried with Na₂SO₄, filtered and evaporated to dryness. The residue was purified with silica column to afford B1 (0.2 g, 74%) as yellow oil.

¹H NMR (400 MHz, Chloroform-d) 63.22-3.12 (m, 4H), 2.36-2.22 (m 4H).

### Synthesis of Intermediate B2

### tert-butyl 1-imino-1l6-thiomorpholine-4-carboxylate 1-oxide

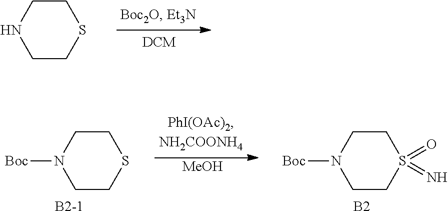

Step 1: To a solution of thiomorpholine (0.18 mL, 1.94 mmol), Et₃N(0.81 mL, 5.82 mmol) in DCM (10 mL) was Boc₂O (634.5 mg, 2.91 mmol). The mixture was stirred at room temperature for 12 hours. The mixture was quenched with water (10 mL) and then extracted with DCM (50 mL*3) The organic layer was combined, washed with brine, dried with Na₂SO₄, filtered and evaporated to dryness. The residue was purified with silica column to afford B1 (0.37 g, 94%) as yellow oil.

¹H NMR (400 MHz, Chloroform-d) δ 3.80-3.60 (m, 4H), 2.72-2.49 (m, 4H), 1.49 (s, 9H).

Step 2: according to the procedure of intermediate B1, started with B2-1 then B2 was yield.

¹H NMR (400 MHz, Chloroform-d) δ 5.18 (s, 1H), 4.09-3.92 (m, 2H), 3.91-3.78 (m, 2H), 3.15-2.98 (m, 4H), 1.49 (s, 9H).

### Synthesis of Compound AA According to Patent (WO2020081999, A109)

### N-(4-((4-(2-(3-chloro-4-(2-chloroethoxy)-5-cyanophenyl) propan-2-yl) phenoxy) methyl) pyrimidin-2-yl) methane sulfonamide

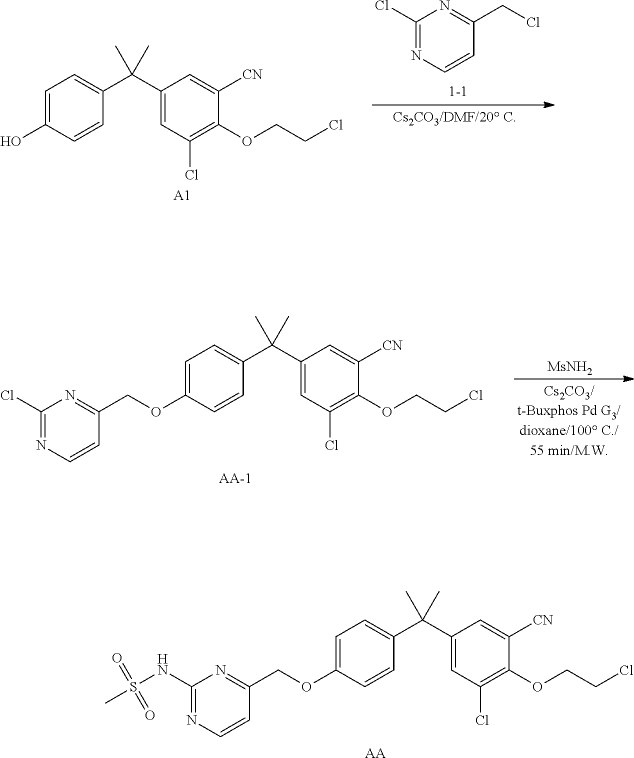

Step 1: To the mixture of compound A1 (1.5 g, 4.28 mmol) and compound 1-1 (698 mg, 4.28 mmol, 1.0 eq) in MeCN (40 mL) was added Cs₂CO₃ (2.79 g, 8.57 mmol, 2.0 eq). Then the mixture was stirred for 2 hours at 40° C. The mixture was filtered and the filtration was concentrated under reduced pressure. The residue was purified by silica (hexane/EtOAc=4/1) to afford the product AA-1 (0.8 g, yield: 39%).

Step 2: To the mixture of AA-1 (800 mg, 1.68 mmol), methanesulfonamide (1.6 g, 16.8 mmol) and Cs₂CO₃ (1.64 g, 5.04 mmol) in dioxane (15 mL) was added t-Buxphos Pd G3 (14.5 mg, 0.025 mmol) in a sealed tube. Then the resulting mixture was stirred for 55 min at 100° C. in Microwave under N₂ atmosphere. After cooling to room temperature, the mixture was filtered and the filtration was concentrated. The residue was diluted with EtOAc (100 mL) and water (100 mL). The organic layer was dried over Na₂SO₄ and then concentrated under reduced pressure. The residue was purified by silica (hexane/EtOAc=1/1) to afford the crude product (600 mg). The crude product was purified by pre-HPLC to afford the desired product N-(4-((4-(2-(3-chloro-4-(2-chloroethoxy)-5-cyanophenyl)propan-2-yl)phenoxy) methyl) pyrimidin-2-yl)methanesulfonamide (compound AA, 253 mg, yield: 28.2%) as white solid.

¹H NMR (400 MHz, Chloroform-d) δ 8.62 (d, J=5.1 Hz, 1H), 7.44 (d, J=2.3 Hz, 1H), 7.31 (d, J=2.4 Hz, 1H), 7.28 (d, J=4.9 Hz, 1H), 7.12 (d, J=8.6 Hz, 2H), 6.90 (d, J=8.6 Hz, 2H), 5.10 (s, 2H), 4.42 (t, J=6.2 Hz, 2H), 3.87 (t, J=6.2 Hz, 2H), 3.47 (s, 3H), 1.64 (s, 6H).

LCMS [M+1]⁺=535.1

### Compound 2

### 3-chloro-2-(2-chloroethoxy)-5-(2-(4-((2-((1-oxidotetrahydro-1λ⁶-thiophen-1-ylidene) amino) pyrimidin-4-yl) methoxy) phenyl) propan-2-yl) benzonitrile

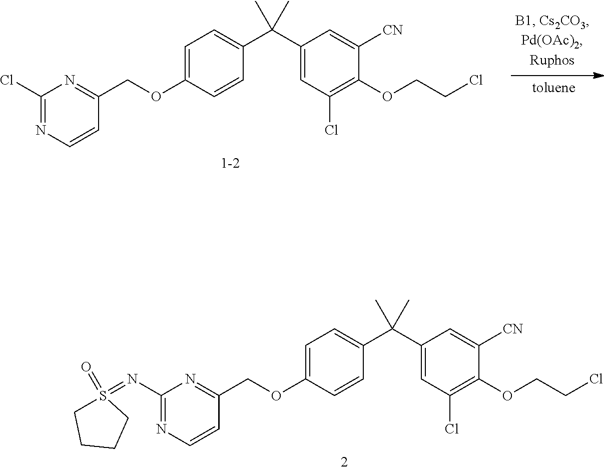 [SMILES: CC(C)(c1ccc(OCc2ccnc(Cl)n2)cc1)c1cc(Cl)c(OCCCl)c(C#N)c1 ; "1-2" ; CC(C)(c1ccc(OCc2ccnc(N=S3(=O)CCCC3)n2)cc1)c1cc(Cl)c(OCCCl)c(C#N)c1 ; "2"]

To the mixture of compound 1-2 (60 mg, 0.088 mmol), compound B1 (21.0 mg, 0.176 mmol), Pd(OAc)₂ (1.98 mg, 0.009 mmol) and Ruphos (8.22 mg, 0.018 mmol) in toluene (1 mL) was added Cs₂CO₃ (43.05 mg, 0.132 mmol). Then the mixture was stirred for 12 hours at 100° C. After cooling to room temperature, the mixture was filtered and the filtration was concentrated. The residue was then diluted with EtOAc (50 mL) and water (30 mL). Then the EtOAc layer was washed by brine (20 mL) and dried over Na₂SO₄. The residue was purified by pre-HPLC to afford the product 3-chloro-2-(2-chloroethoxy)-5-(2-(4-((2-((1-oxidotetrahydro-1λ⁶-thiophen-1-ylidene) amino) pyrimidin-4-yl)methoxy)phenyl)propan-2-yl)benzonitrile (compound 2, 5 mg, yield: 10.14%).

¹H NMR (400 MHz, DMSO-d6) δ 8.51-8.40 (m, 1H), 7.70-7.54 (m, 2H), 7.23-7.13 (m, 2H), 7.00-6.97 (m, 3H), 5.03 (s, 2H), 4.49-4.36 (m, 2H), 3.99-3.91 (m, 2H), 2.29-1.98 (m, 4H), 1.71-1.57 (m, 4H), 1.24 (s, 6H).

LCMS [M+H]⁺=559.1

### Compound 6

### 3-chloro-2-(2-chloroethoxy)-5-(2-(4-((2-((1-oxido-1λ⁶-thiomorpholin-1-ylidene) amino)pyrimidin-4-yl)methoxy)phenyl)propan-2-yl)benzonitrile

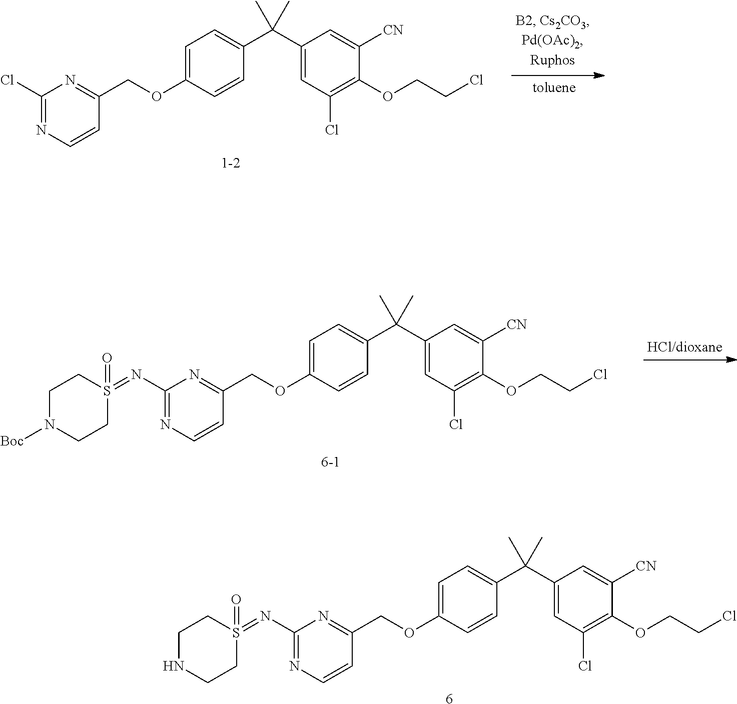 [SMILES: CC(C)(c1ccc(OCc2ccnc(Cl)n2)cc1)c1cc(Cl)c(OCCCl)c(C#N)c1 ; "1-2" ; CC(C)(C)OC(=O)N1CCS(=O)(=Nc2nccc(COc3ccc(C(C)(C)c4cc(Cl)c(OCCCl)c(C#N)c4)cc3)n2)CC1 ; "6-1" ; CC(C)(c1ccc(OCc2ccnc(N=S3(=O)CCNCC3)n2)cc1)c1cc(Cl)c(OCCCl)c(C#N)c1 ; "6"]

Step 1: To the mixture of compound 1-2 (50 mg, 0.105 mmol), compound B2 (24.60 mg, 0.105 mmol), Ruphos (9.79 mg, 0.021 mmol) and Cs₂CO₃ (51.25 mg, 0.157 mmol) in toluene (1 mL) was added Pd(OAc)₂ (2.36 mg, 0.01 mmol). Then the mixture was stirred for 12 hours at 100° C. under N₂ atmosphere. After cooling to room temperature, the mixture was diluted with water (20 mL) and then extracted with EtOAc (30 mL*2). The EtOAc layer was washed by brine (30 mL) and then dried over Na₂SO₄. The residue was purified by silica (DCM/MeOH=25/1) to afford the product 3-1 (40 mg, yield: 56.47%).

LCMS [M+H-Boc]⁺=574.1

Step 2: The mixture of compound 3-1 (40 mg, 0.06 mmol) in DCM (1 mL) and HCl/dioxane (1 mL, 4 M) was stirred for 12 hours at room temperature. Then the mixture was concentrated and the residue was purified by pre-HPLC to afford the product 3-chloro-2-(2-chloroethoxy)-5-(2-(4-((2-((1-oxido-1λ⁶-thiomorpholin-1-ylidene)amino)pyrimidin-4-yl)methoxy)phenyl)propan-2-yl)benzonitrile (compound 3, 30 mg, yield: 82.81%).

¹H NMR (400 MHz, MeOD-d4) δ 8.45 (d, J=5.2 Hz, 1H), 7.50 (dd, J=18.0, 2.4 Hz, 2H), 7.19 (d, J=8.8 Hz, 2H), 7.10 (s, 1H), 6.95 (d, J=8.8 Hz, 2H), 5.08 (s, 2H), 4.46-4.36 (m, 2H), 3.90 (t, J=5.6 Hz, 2H), 3.87-3.76 (m, 2H), 3.54-3.37 (m, 4H), 3.36-3.32 (m, 2H), 1.67 (s, 6H).

LCMS [M+H]⁺=574.1

### Compound 60

### N-(5-((4-(2-(3-chloro-4-(2-chloroethoxy)-5-cyanophenyl)propan-2-yl)phenyl)ethynyl)pyrimidin-2-yl)methanesulfonamide

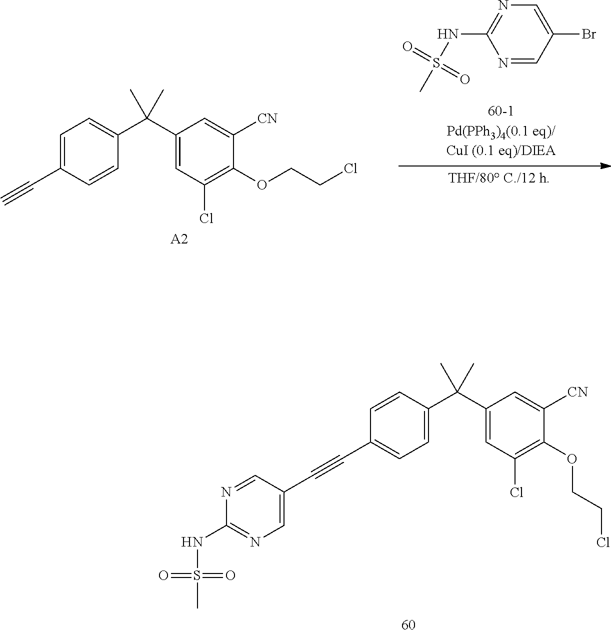

To the mixture of compound A2 (2.3 g, 5.3 mmol), compound 4-1 (1.3 g, 5.3 mmol) and DIEA (13.7 g, 106 mmol) in THE (13 ml) was added Pd(PPh₃)₄ (613 mg, 6.4 mmol) and CuI (101 mg, 0.53 mmol). The mixture was then stirred for 16 hours at 80° C. under N₂ atmosphere. After cooling to room temperature, the mixture was diluted with water (50 mL) and then extracted with EtOAc (50 mL*3). The EtOAc was washed by brine (50 mL) and dried over Na₂SO₄. Then the organic layer was concentrated under reduced pressure. The residue was purified by silica (hexane/EtOAc=1/1) to afford the crude product. The crude product was purified again by pre-HPLC to afford the product N-(5-((4-(2-(3-chloro-4-(2-chloroethoxy)-5-cyanophenyl)propan-2-yl)phenyl)ethynyl)pyrimidin-2-yl)methanesulfonamide (compound 4, 253 mg, yield: 7.4%) as white solid.

¹H NMR (400 MHz, Chloroform-d) δ 8.74 (s, 2H), 7.50 (d, J=7.7 Hz, 2H), 7.43 (s, 1H), 7.34 (d, J=2.3 Hz, 1H), 7.20 (d, J=8.2 Hz, 2H), 4.44 (t, J=6.3 Hz, 2H), 3.88 (t, J=6.2 Hz, 2H), 3.49 (s, 3H), 1.68 (s, 6H).

LC-MS[M+1]⁺=529.1

### Compound 61: The Synthesis of Compound 61

### 3-chloro-5-(2-(4-((2-((1-oxidotetrahydro-1λ⁶-thiophen-1-ylidene)amino)pyrimidin-5-yl)ethynyl)phenyl)propan-2-yl)-2-(oxiran-2-ylmethoxy)benzonitrile

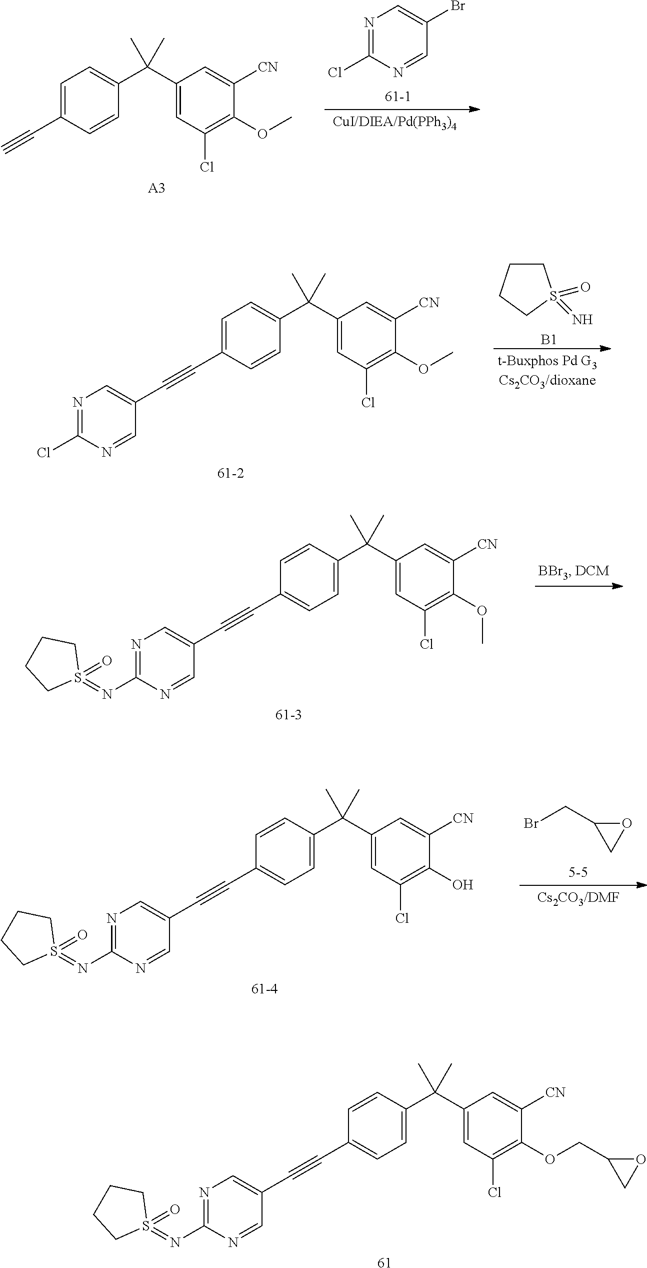

Step 1: To the mixture of A3 (1.5 g, 4.84 mmol) and compound 61-1 (1.03 g, 5.33 mmol) in THE (10 mL) was added CuI (276 mg, 1.45 mmol), DIEA (3.12 g, 24.2 mmol) and Pd(PPh₃)₄ (839 mg, 0.726 mmol). Then the mixture was stirred for 2 hours at 80° C. under N₂ atmosphere in Microwave. After cooling to room temperature, the mixture was concentrated and the residue was diluted with water (50 mL) and then extracted with EtOAc (50 mL*3). The EtOAc was washed by brine (50 mL) and dried over Na₂SO₄. Then the organic layer was concentrated under reduced pressure. The residue was purified by silica (hexane/EtOAc=3/1) to afford the product 61-2 (1.1 g, yield: 53.8%) as yellow solid.

Step 2: To the mixture of compound 61-2 (0.5 g, 1.18 mmol), compound B1 (141 mg, 1.18 mmol) and t-Buxphos Pd G₃ (93.8 mg, 0.12 mmol) in dioxane (7 mL) was added and Cs₂CO₃ (964 mg, 2.96 mmol). Then the mixture was stirred for 1 hour at 100° C. in Microwave under N₂ atmosphere. After cooling to room temperature, the mixture was filtered and the filtration was concentrated. The residue was then diluted with EtOAc (50 mL) and water (30 mL). Then the EtOAc layer was washed by brine (30 mL) and dried over Na₂SO₄. The EtOAc was concentrated and the residue was purified by silica (hexane/EtOAc=1/5) to afford the product 61-3 (470 mg, yield: 82.5%) as yellow solid.

LC-MS[M+1]⁺=505.1

Step 3: To the mixture of 61-3 (230 mg, 0.39 mmol) in DCM (4 mL) was added dropwise BBr₃ (198 mg, 0.79 mmol). The mixture was then stirred for 16 hours at room temperature. Then the mixture was quenched by sat. NH₄Cl (10 mL) and extracted with DCM (10 mL*2). The DCM layer was washed by brine (30 mL) and dried over Na₂SO₄. The DCM was then concentrated to afford the crude product 5-4 (200 mg, yield: 89%) as yellow oil.

LC-MS[M+1]⁺=491.1

Step 4: To the mixture of compound 61-4 (180 mg, 0.38 mmol) and Cs₂CO₃ (315 mg, 0.97 mmol) in DMF (4 mL) was added compound 5-5 (132 mg, 0.97 mmol). The mixture was stirred for 16 hours at 70° C. After cooling to room temperature, the residue was then diluted with EtOAc (50 mL) and water (30 mL). Then the EtOAc layer was washed by brine (30 mL*2) and dried over Na₂SO₄. The EtOAc was concentrated and the residue was purified by silica (hexane/EtOAc=1/5) to afford the product 3-chloro-5-(2-(4-((2-((1-oxidotetrahydro-1λ⁶-thiophen-1-ylidene)amino) pyrimidin-5-yl)ethynyl)phenyl)propan-2-yl)-2-(oxiran-2-ylmethoxy)benzonitrile (compound 61, 70 mg, yield: 33%) as white solid.

¹H NMR (400 MHz, Chloroform-d) δ 8.59 (s, 2H), 7.45 (d, J=8.6 Hz, 2H), 7.40 (d, J=2.4 Hz, 1H), 7.33 (d, J=2.4 Hz, 1H), 7.16 (d, J=8.6 Hz, 2H), 4.40-4.36 (m, 3.5 Hz, 1H), 4.19-4.14 (m, 1H), 3.71-3.64 (m, 2H), 3.46-3.37 (m, 3H), 2.92-2.86 (m, 1H), 2.76-2.71 (m, 1H), 2.42-2.21 (m, 4H), 1.65 (s, 6H).

LC-MS[M+1]⁺=547.2

### Compound 62: Synthesis of Compound 62

### 3-chloro-2-(2-hydroxyethoxy)-5-(2-(4-((2-((1-oxido-1λ⁶-thiomorpholin-1-ylidene)amino)pyrimidin-4-yl)methoxy)phenyl)propan-2-yl)benzonitrile

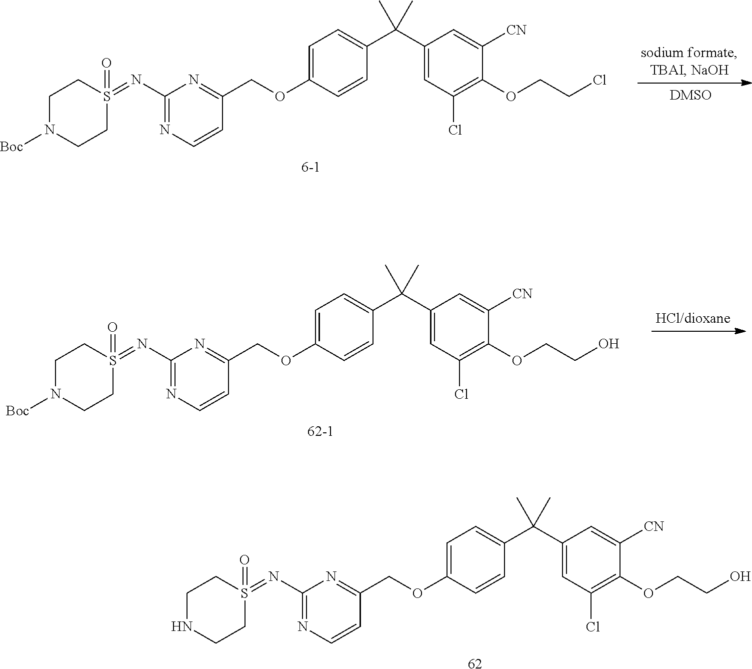 [SMILES: CC(C)(C)OC(=O)N1CCS(=O)(=Nc2nccc(COc3ccc(C(C)(C)c4cc(Cl)c(OCCCl)c(C#N)c4)cc3)n2)CC1 ; "6-1" ; CC(C)(C)OC(=O)N1CCS(=O)(=Nc2nccc(COc3ccc(C(C)(C)c4cc(Cl)c(OCCO)c(C#N)c4)cc3)n2)CC1 ; "62-1" ; CC(C)(c1ccc(OCc2ccnc(N=S3(=O)CCNCC3)n2)cc1)c1cc(Cl)c(OCCO)c(C#N)c1 ; "62"]

Step 1: A solution of compound 6-1 (100 mg, 0.148 mmol), sodium formate (25.20 mg, 0.371 mmol) and tetrabutylazanium iodide (5.47 mg, 0.015 mmol) in DMSO (1 mL) was stirred at 110° C. for 2 hours. The reaction was cooled to 25° C. and NaOH (11.86 mg, 0.296 mmol) was added. The mixture was then stirred at 25° C. for 12 hours. The mixture was extracted with water (10 mL) and DCM (5 mL*3). The organic layer was washed with brine (10 mL), dried and filtered. The filtrate was concentrated under reduced pressure. The residue was purified by silica (0-5% MeOH/DCM) to afford compound 62-1 (50 mg, 0.076 mmol, 51.41%) as white solid.

LCMS [M+H]⁺=656.2

Step 2: The mixture of compound 62-1 (50 mg, 0.08 mmol) in DCM (1 mL) and HCl/dioxane (1 mL, 4 M) was stirred for 12 hours at room temperature. Then the mixture was concentrated and the residue was purified by pre-HPLC to afford the compound 62 (15 mg, yield: 35.4%).

1H NMR (400 MHz, MeOD-d4) δ 8.43 (d, J=5.2 Hz, 1H), 7.48 (dd, J=17.6, 2.4 Hz, 2H), 7.19-7.15 (m, 2H), 7.07 (d, J=5.2 Hz, 1H), 7.01-6.91 (m, 2H), 5.07 (s, 2H), 4.24 (t, J=4.8 Hz, 2H), 3.91 (t, J=4.8 Hz, 2H), 3.84-3.71 (m, 2H), 3.52-3.32 (m, 4H), 3.21 (ddd, J=14.0, 8.8, 2.8 Hz, 2H), 1.65 (s, 6H).

LCMS [M+H]⁺=556.1

### Example 2: Live Cell Imaging of the Nuclear Distribution of Androgen Receptor (AR)

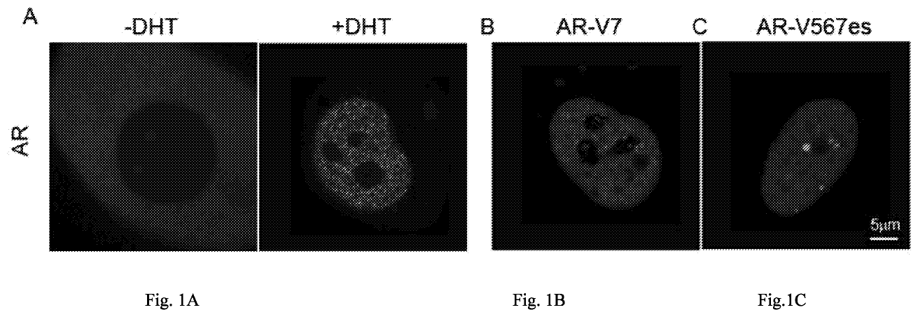

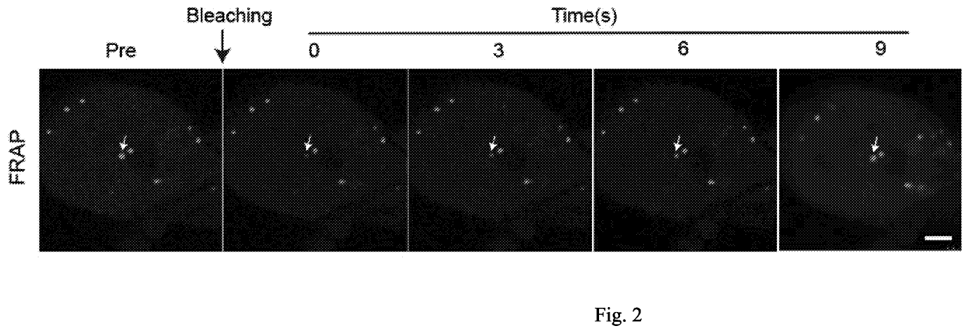

Androgen receptor (AR) belongs to the family of steroid hormone receptor family and could be specifically activated by corresponding ligand (such as dihydrotestosterone, DHT). To investigate the subnuclear distribution of active AR, we stably expressed enhanced green fluorescent protein (mEGFP) labeled AR in prostate cancer cell LNCaP. Live-cell imaging (see FIG. 1A-1C) revealed that unliganded AR (FIG. 1A, −DHT, left) primarily located in the cytoplasm, while after DHT binding (FIG. 1A, +DHT, right), AR translocated into nucleus and displayed a non-uniform distribution. To further interrogate the distribution of constitutively active AR splice variants associated with castration-resistant prostate cancers (CRPCs), we performed live-cell imaging with LNCaP cells transfected with AR-V7 (FIG. 1B) and AR-V567es (FIG. 1C) tagged with mEGFP. Both of these two active splice variants exhibited a discrete nuclear distribution. These together suggest that transcriptionally active AR could form discrete nuclear puncta.

The specific method and results are as follows:

Live-Cell Imaging Method

Cells were grown on 24-well glass bottom plate (Cellvis, P24-1.5H-N) and images were taken with the Leica TCS SP8 confocal microscopy system using a 100× oil objective (NA=1.4). Cells were imaged on a heated stage (37° C.) and supplemented with warmed (37° C.) humidified air. The microscope was enclosed in an incubation chamber heated to 37° C.

LNCaP cells were transfected with an expression vector for AR-mEGFP using steroid hormone-free and phenol red-free RPMI media for 48 hours before adding 1 nM DHT. For LNCaP cells transfected with ligand-independent splice variants AR V7 and AR V567es, cells were cultured with normal RPMI media and DHT stimulation is not needed

For compounds treatment assay, LNCaP cells were transfected with an expression vector for AR (AR or AR F877L/T878A)-mEGFP using steroid hormone-free and phenol red-free RPMI media for 24 hours before compound treatment. After 10 μM compounds treatment for 3 hour, LNCaP cells were added with 1 nM DHT. For ligand-independent AR V7-mEGFP expressing LNCaP cells, starvation step is skipped and compounds were added 12 hours after transfection. The final concentration of DMSO was 0.1%. Cells were subjected to microscopic observation every 30 mins after DHT/compounds were added.

Data was analyzed by images collected from 100 representative fields in each group. The spots quantification was performed based on the area and intensity of the spots through green (AR-mEGFP) channel. Fluorescent images were processed and assembled into figures using LAS X (Leica) and Fiji. GraphPad Prism is used to plot and analyze the compound treatment results.

### Example 3: Fluorescence Recovery after Photobleaching (FRAP) of the Nuclear AR Puncta

Liquid-liquid phase separation has emerged as the fundamental mechanism of biological processes and mounting evidences corroborate gene regulation occurs in transcriptional condensates. Next we assessed whether the nuclear puncta of active AR display liquid-like features. Fluorescence recovery after photobleaching (FRAP) experiments showed that upon bleaching, the fluorescence of the DHT-stimulated AR-mEGFP recovered within seconds. This result indicates that activated AR (Androgen receptor) formed nuclear puncta (i.e. condensate) with liquid-like properties.

FRAP assay was conducted using the FRAP module of the Leica SP8 confocal microscopy system. The AR-mEGFP were bleached using a 488-nm laser beam, respectively. Bleaching was focused on a circular region of interest (ROI) using 100% laser power and time-lapse images were collected. The live-cell imaging methods are the same as described in Example 2.

### Example 4: Immunofluorescence with AR Condensates

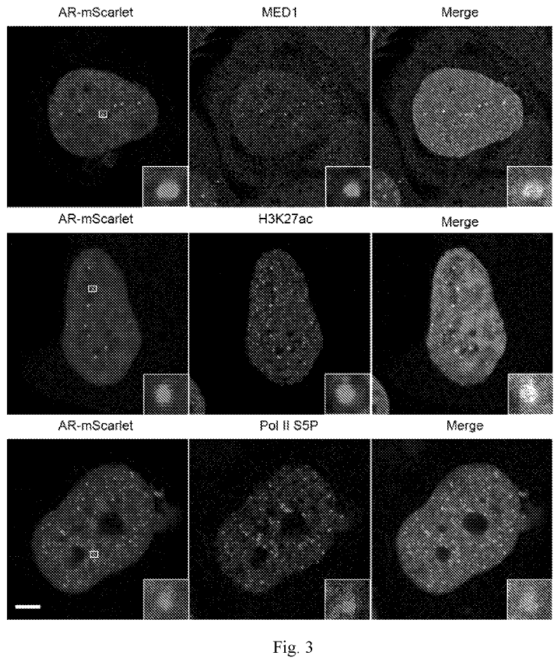

To investigate whether AR nuclear condensates were active-transcription regions, we analyzed the colocation of AR puncta and several active transcription markers by immunofluorescence. The AR puncta were enriched for super-enhancer marker MED1, actively transcribed chromatin mark H3K27ac and the active RNA polymerase II phosphorylated at Ser5 of its C-terminal domain. These data support that AR forms LLPS condensates that are sites of active transcription (FIG. 3).

After DHT stimulation, H1299 cells expressing AR-mScarlet were fixed with 4% PFA for 15 minutes at room temperature. Cells were subsequently washed with PBS (3×3 min) and blocked in PBS/BSA (3%)/Triton X-100 (0.3%) at room temperature for 60 minutes. Cells were rinsed in PBS and incubated with anti-MED1 (ab64965, abcam) anti-H3K27ac (ab4729, Abcam), anti-RNA Pol II-S5P antibody (#04-1572, Millipore) in PBS/BSA (1%) overnight at 4° C. Cells were washed with PBST (1×PBS+Tween20 0.2%, 3×5 min) and incubated with the secondary antibody for 1 hours at room temperature. After washing with PBST (3×5 min), cells were stained with DAPI. Images were taken with the Leica TCS SP8 confocal microscopy system using a 100× oil objective (NA=1.4) and further post-processed using FIJI.

As shown in FIG. 3, co-localization was observed for super-enhancer marker MED1 (green), actively transcribed chromatin mark H3K27ac (green) and the active RNA polymerase II phosphorylated at Ser5 of its CTD (green) with AR-mScarlet (red) puncta in H1299 cells.

### Example 5: Androgen Receptor N-Terminal Domain (AR-NTD) is Responsible for AR Liquid-Liquid Phase Separation (LLPS)

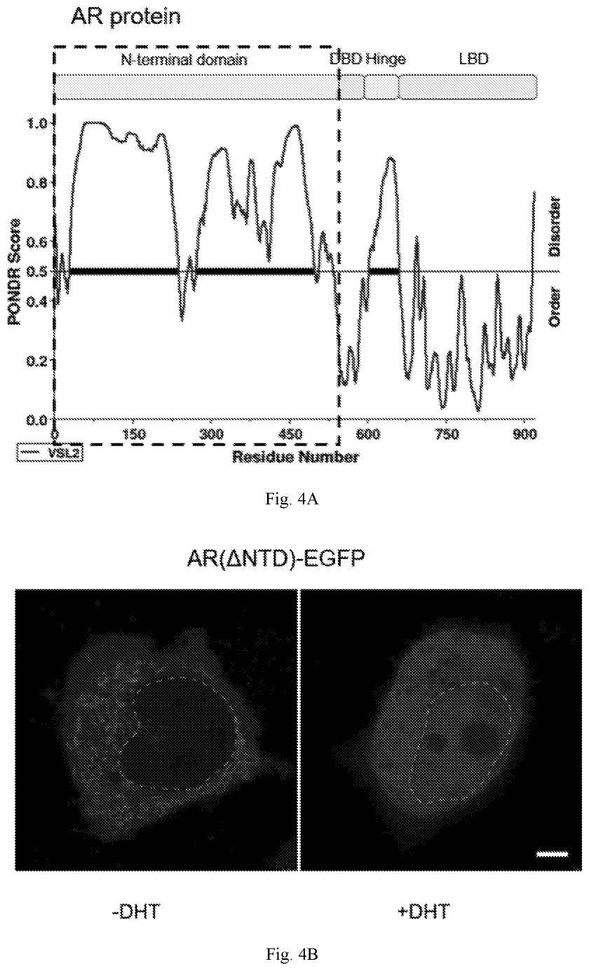

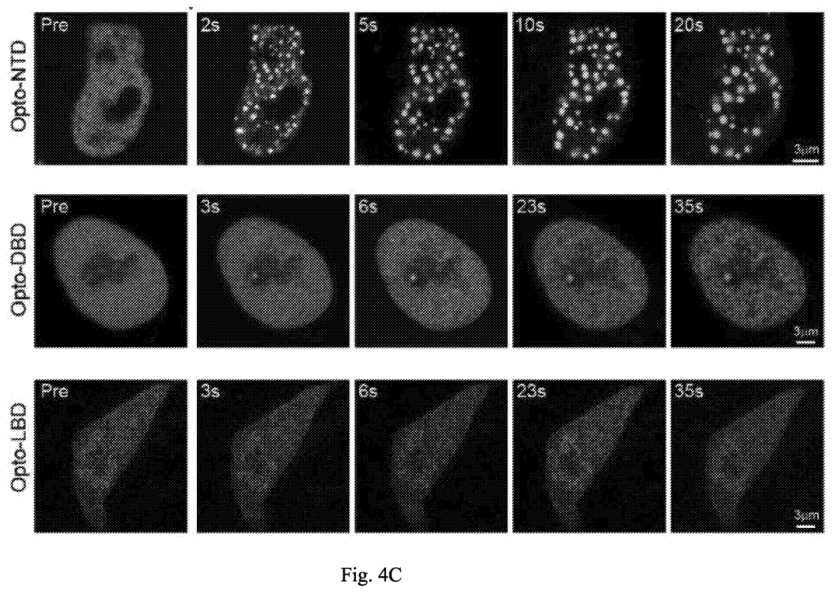

Full length AR is composed of an intrinsically disordered NTD, a folded DNA-binding domain (DBD), a disordered hinge region and a folded ligand binding domain (LBD). Bioinformatics analysis predicted that AR-NTD is unstructured and contains large intrinsic disordered regions (IDRs) (FIG. 4A). We investigated whether AR-NTD is necessary for AR phase separation. Live-cell imaging revealed that deletion of NTD abolished the LLPS capability of AR (FIG. 4B). To further explore if AR-NTD participates in driving LLPS, we used a previously reported optoIDR assay to test IDR-dependent, light-activated puncta formation. The N-terminal domain of AR is fused to a photo-activatable, self-associating Cry2 protein labeled with mCherry.

The OptoIDR assay was performed as follows.

AR NTD, DBD and LBD were cloned into lentiviral backbone containing mCherry-Cry2 fusion protein with SV40 NLS. LNCaP cells were plated one day before the transduction and plasmids were transduced using jetOPTINIUS(Polyplus, 117-15) according to the manufacturer's instructions. Images were taken with the Leica TCS SP8 confocal microscopy system using a 100× oil objective (NA=1.4). Cells were imaged on a heated stage(37° C.) and supplemented with warmed (37° C.) humidified air. Puncta formation was induced with 488 nm light pulses, mCherry fluorescence was stimulated with 561 nm light. The live-cell imaging methods are the same as described in example 2.

FIG. 4C shows representative images of blue-light induced clustering of AR(NTD), AR(DBD) and AR(LBD)-CRY2-SV40 NLS at different time points in LNCaP cells. Cells were stimulated with 488 nm laser.

Time-lapse imaging showed that fusion of AR-NTD to Cry2-mCherry promoted the rapid formation of spherical droplets stimulated by blue light, while the DBD and LBD exhibited diffuse distribution. These together suggest that the intrinsically disordered AR-NTD is responsible for driving AR phase separation.

### Example 6: Characterization of Compounds Modulating AR Condensates

Enzalutamide is known to be an AR antagonist which inhibits androgen binding to AR.

Given that AR puncta are sites of active transcription and AR is the driver oncogene in prostate cancer, we explore the possibility that compounds impairing AR condensates could suppress AR transcriptional activity and prostate cancer growth.

LNCaP cells expressing AR (AR or AR F877L/T878A or V7)-mEGFP were cultured in steroid hormone-free and phenol red-free RPMI media for 24 hours before treatment with or without 10 μM test compound (e.g., Enzalutamide). After 10 μM compounds treatment for 3 hour, LNCaP cells were added with 1 nM DHT. For ligand-independent AR V7-mEGFP expressing LNCaP cells, starvation step is skipped and compounds were added 12 hours after transfection. The live-cell imaging methods are the same as described in Example 2.

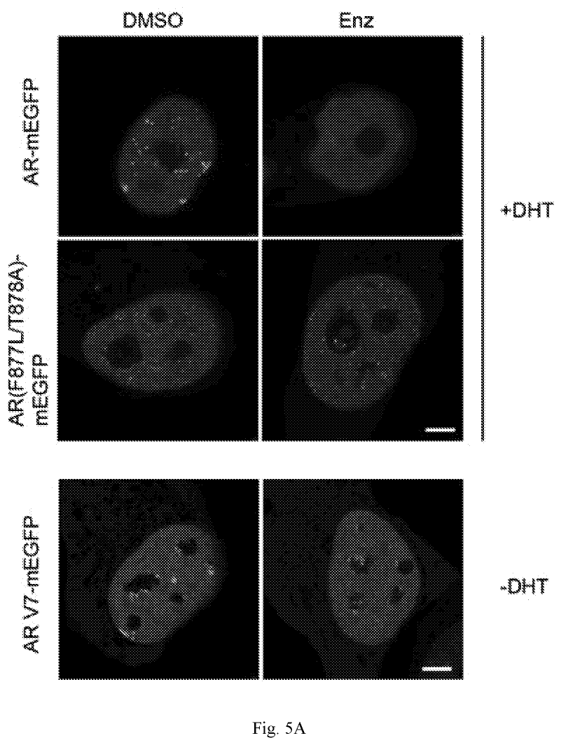

FIG. 5A shows live-cell imaging showing LNCaP cells expressing AR (AR or AR F877L/T878A or V7)-mEGFP treated with or without 10 μM Enzalutamide. Microscopic images showed that Enzalutamide indeed abolished the puncta formation of AR, while exerted no effects on its resistant mutant F877L/T877A. AR V7 is a constitutive active variant lacking LBD and has been reported to be a major resistance mechanism to Enzalutamide. As shown in FIG. 5A, Enzalutamide can barely impair the puncta formation of AR V7. This result suggests that AR activity is well correlated with the capability of AR phase separation.

We tested several compounds synthesized in the present disclosure. Among these compounds, we found that Compound 60 could suppress the phase separation of both AR and Enzalutamide resistant mutants such as F877L/T877A mutant and AR V7 mutant (FIG. 5B).

FIG. 5B shows live-cell imaging showing LNCaP cells expressing AR (AR or AR F877L/T878A or V7)-mEGFP treated with or without 10 μM Compound 60. The live-cell imaging methods are the same as described in Example 2. As shown in FIG. 5B, Compound 60 abolished the puncta formation of not only AR, but also its resistant mutants such as F877L/T877A mutant and AR V7 mutant. This result suggests that inhibition of LLPS formation can overcome resistance of AR mutation.

### Example 7: Characterization of One or More Compounds in AF-1 NMR Binding Assay

Binding of the exemplary compounds to human AR-NTD was performed using STD Saturation Transfer Diffusion Nuclear Magnetic Resonance (STD NMR) analysis with purified AF-1 recombinant protein.

Method:

Binding of the exemplary compounds to human AR-NTD was performed using STD Saturation Transfer Diffusion Nuclear Magnetic Resonance (STD NMR) analysis with purified Tau-5AF-1 recombinant protein. All NMR spectroscopy experiments were performed on a Bruker Avance III 600 MHz spectrometer. NMR samples contain 25 μM AF-1 and 250 μM compound, and were diluted by PBS (pH 7.4, 60% D20) buffer to 500 μL. For the STD experiments, the standard Bruker stddiffgp19.3 pulse sequence was used with a saturation time of 2 s and a spectral width of 16 ppm with 128 scans. The on-resonance frequency was set to −0.4 ppm, while the off-resonance frequency was set to 33 ppm. Appropriate blank experiments, in the absence of protein or compound, were performed to test the lack of direct saturation to the compound protons.

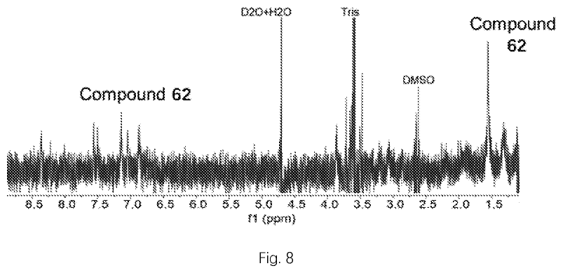

In STD NMR, binding results in a difference spectrum with peaks corresponding to compound, whereas if no binding occurs, the difference spectrum does not show any peaks. As shown in FIG. 8, the clear signals observed in the STD difference spectrum of AF-1 protein in the presence of 10 molar equivalents Compound 62 confirmed the binding between AF-1 and the compound.

### Example 8: Mass Spectrometry Analysis of Compound 61 and Protein Adducts

50 μg of recombinant AR truncated proteins (AF-1) was incubated with 20 μM Compound 61 in Tris-NaCl buffer at R.T. for 2 hours. Combine sample with digest buffer in the filter unit, and mix with 3 μL Trypsin enzyme to digest at 70° C. for 1.5 hours. The 100 μL proteins were reduced by adding 2 μL 1M DTT by 37° C. 30 min and alkylated with 5.5 μL 1M iodoacetamide in darkness for 30 min at R.T. Then digests were stopped with 0.5% formic acid. After syringe filtration through 22 μm filters, the clear solution was subjected to LC-MS/MS separation. Each sample was desalted by loading on a Thermo C18 PepMap100 precolumn (300 μm×5 mm) and eluted on a Thermo Acclaim PepMap RSLC analytical column (75 μm×15 cm). Mobile phase A (0.1% formic acid in H2O) and mobile phase B (0.1% formic acid in acetonitrile) were used to establish gradient elution process. The flow rate was 0.3 μL/min. The original mass spectrum was processed by Waters UNIFI. The binding sites of compound are found to be within the NTD of AR protein.

The AF-1 protein and compound 61 adduct was detected by Intact Mass, and 3 binding sites were identified by peptide mapping (Table 1).

TABLE 1
Binding sites of compound 61 identified on
AF-1 protein

| | | | Rate |
|---|---|---|---|
| Peptide | Site | M.W. | (%) |
| GDC(+546.15)MYAPLLGVPPAVRPTPCA | C267 | 3314.50 | 2.0 |
| PLAECK | | | |
| GLEGESLGC(+546.15)SGSAAAGSSGTL | C327 | 3487.57 | 0.6 |
| ELPSTLSLYK | | | |
| LENPLDYGSAWAAAAAQC(+546.15)R | C406 | 2552.08 | 1.7 |

### Example 9 Characterization of One or More Biotinylated Compounds on Tau-5 Binding Via a Pull Down Assay

To confirm whether the exemplary compounds bind to Tau-5 region of AR protein, a cell-free binding assay was performed with purified AR Tau-5 recombinant protein tagged with EGFP.

Method

Recombinant AR Tau-5 was mixed with alkyne-containing test compounds at 4° C. overnight. Then compounds were labeled with biotin by Click-chemistry reaction at 25° C. for 3 hours in buffer containing 0.1% SDS, 5% t-butanol, 100 μM tris[(1-benzyl-1H-1,2,3-triazol-4-yl)methyl]amine, 1 mM tris-(2-carboxyethyl) phosphine (TCEP), 100 μM biotin-azide reagent, and 1 mM CuSO4. Samples were dialyzed overnight in 50 mM HEPES (pH 8.0), 150 mM NaCl, 0.1% SDS, and 1% Triton-X100 to remove excess biotin-azide reagent. Biotinylated compounds bound to Tau-5-GFP proteins were enriched using streptavidin-agarose resin. Biotin-compound-protein complexes were resolved on SDS-PAGE and subjected to Western blot analysis using anti-GFP antibody.

### Example 10 Characterization of a Set of Compounds in ARE-Luciferase Reporter Assay

The ARE-luciferase reporter assay was performed to evaluate whether the exemplary compounds could regulate the transcriptional activity of AR or AR variants in prostate cancer cells.

Method

The ARE-luciferase reporter plasmid contains functional AREs (androgen response elements) to which AR binds in response to androgen to induce luciferase activity. LNCaP (Cobioer) cells were transiently transfected with the ARE-luciferase reporter in charcoal stripped FBS/phenol red-free RPMI medium prior to treatment for 24 hours. Then DMSO or test compounds with serial diluted concentrations were added one hour prior to the addition of DHT(1 nM). After 24 h of incubation with DHT, cells were lysed and luciferase activity was assayed using the ONE-Glo™ Luciferase Assay System (Promega) and measured using EnVision Multimode Plate Reader, according to manufacturer's instructions. Values were normalized to cell viability determined by CellTiter-Glo® Luminescent Cell Viability Assay Kit (Promega). To determine the IC₅₀, treatments were normalized to vehicle control activity induced by DHT, the mean value of vehicle control without DHT treatment was set as 100% inhibition. GraphPad Prism graphing software was used to calculate IC₅₀ values.

To evaluate the transcriptional activity of AR-V7, HEK293T(Cobioer) cells were transiently cotransfected with an expression vector for AR-V7 and the ARE-luciferase reporter in hormone-starved conditions. For 22Rv1(Cobioer) prostate cancer cell line, which expresses endogenous AR-V7, ARE-luciferase reporter was transiently transfected in hormone-starved conditions. 24 h after transfection, DMSO or test compounds with serial diluted concentrations were added for additional 24 h incubation. Reporter activity was detected and normalized by cell viability as described above. Treatments were normalized to DMSO control activity, and IC₅₀ values were further determined via GraphPad Prism graphing software.

The results were listed as below and suggested that the representative compounds could actively inhibit transcriptional activity of either wildtype-AR or AR-V7 variant. Especially in the two AR-V7-expressing cells, the representative compounds showed surprisingly much better activity on suppressing AR-V7 transcriptional activity.

TABLE 2
Summary of inhibitory IC₅₀ of test compounds
in ARE-luciferase reporter assay

| | LNCaP | HEK293T(V7) | 22Rv1 |
|---|---|---|---|
| | ARE-luc | ARE-luc | ARE-luc |
| Compound ID | IC₅₀ (μM) | IC₅₀ (μM) | IC₅₀ (μM) |
| Enzalutamide | 0.249 | >20 | 58 |
| Compound AA | 12.57 | >20 | >20 |
| Compound 2 | 2.82 | 2.3 | 4.4 |
| Compound 6 | 3.5 | 2.1 | 8.6 |
| Compound 60 | 4.7 | 8.9 | |

### Example 11 Characterization of a Set of Compounds in AR/AR-V7 Target Gene qPCR Assay

To evaluate the effect of exemplary compounds on mRNA expression of AR or AR-V7 downstream target genes, real-time quantitative PCR measurements were performed in prostate cancer cell line 22Rv1 expressing both AR and AR-V7.

Method

22Rv1 cells were cultured in charcoal stripped FBS/phenol red-free RPMI medium for 24 h, and then treated with DMSO or test compounds for additional 48 h prior to RT-qPCR. Briefly, RNA was isolated from cells using RNeasy Mini Kit (Qiagen) following the manufacturer's protocol. RNA was reverse transcribed and subjected to qPCR using PowerUp™ SYBR™ Green Master Mix (Applied Biosystems). The relative quantity of the target genes was calculated using the ΔΔCₜ method by comparing Cₜ of the target genes and mean Cₜ of the housekeeping gene GAPDH.

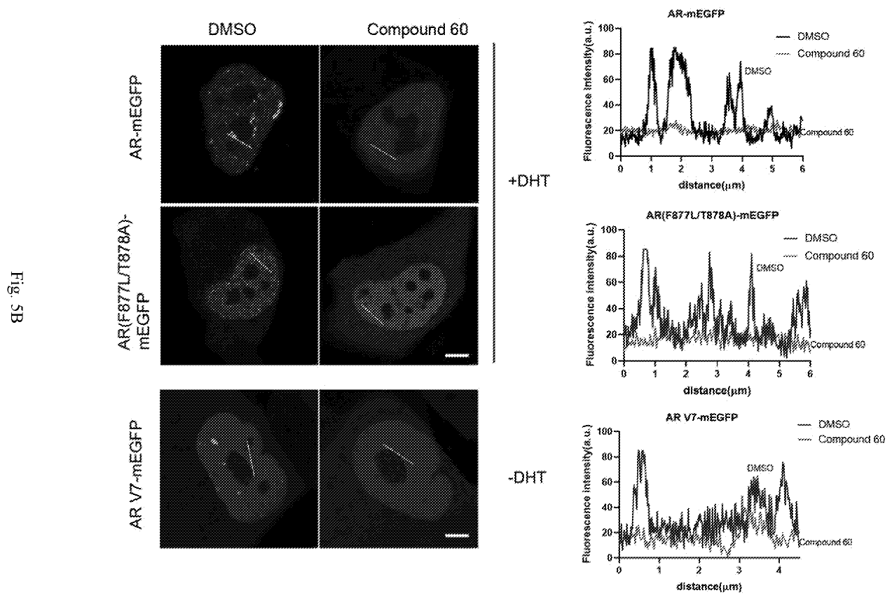

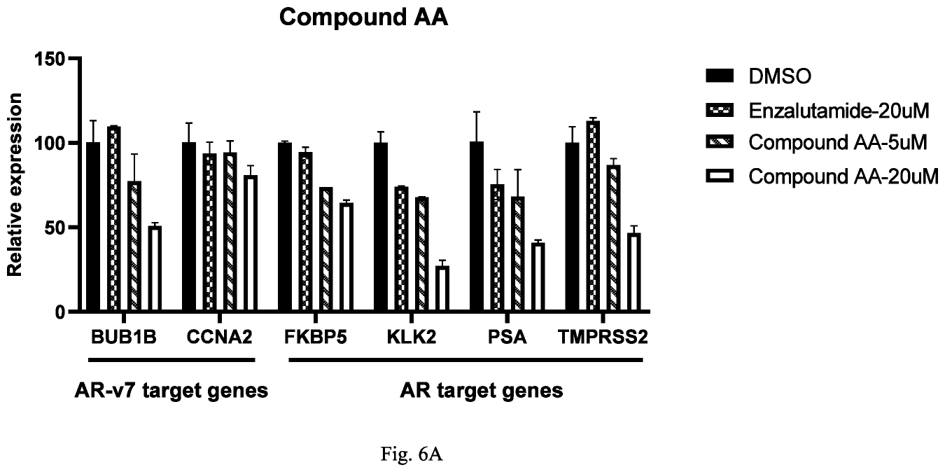

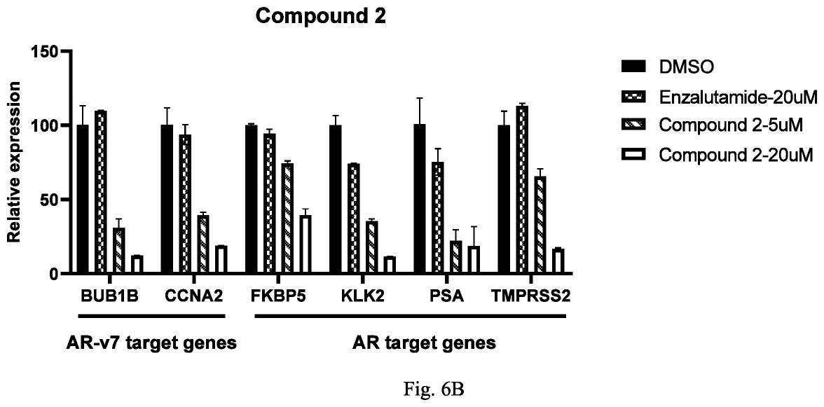

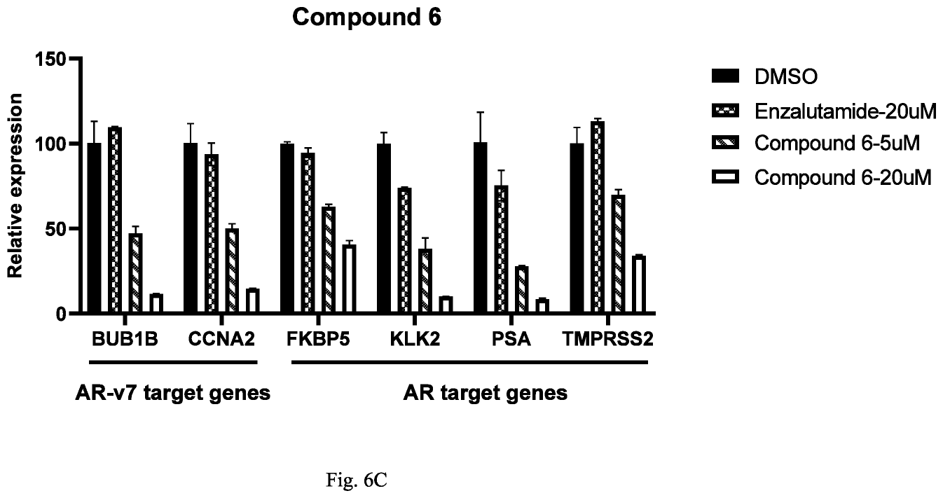

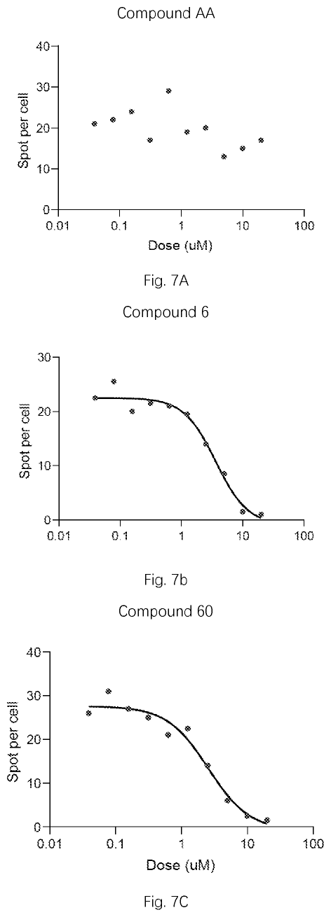

The result suggested that the test compounds could inhibit mRNA expression of target genes of either AR or AR-V7 (FIG. 6A, 6B, 6C).

### Example 12 Characterization of a Set of Compounds in Proliferation Assay in Prostate Cancer Cell Lines

To evaluate the effect of exemplary compounds on proliferation of AR/AR-V7-dependent prostate cancer cells, cell viability assay was performed in LNCaP (AR-positive), 22Rv1 (AR-positive; AR-V7 positive), VCaP (AR-positive; AR-V7 positive), and DU145 (AR-negative) cells, respectively.

Method

LNCaP cells were seeded in charcoal stripped FBS/phenol red-free RPMI medium in 96-well plates for 24 h before pretreating for 1 h with test compounds prior to addition of 0.2 nM DHT for 4 days. 22Rv1 or VCaP cells were cultured in charcoal stripped FBS/phenol red-free RPMI medium for 24 h before addition of test compounds for 4 days. DU145 cells were seeded in complete medium for 24 h before addition of test compounds for 4 days. Cell proliferation was measured with CellTiter-Glo® Luminescent Cell Viability Assay (Promega). In LNCaP cells, DHT-dependent cell growth is calculated by measuring the difference between vehicle control (DMSO) cells treated with or without 0.2 nM DHT. The IC₅₀ values were determined using the four parameter fit via GraphPad Prism graphing software.

The result suggested that the test compounds could suppress AR/AR-V7-dependent prostate cancer cell proliferation.

TABLE 3
IC₅₀ of test compounds in cell proliferation assay

| | | 22Rv1 | VCaP | |
|---|---|---|---|---|
| | LNCaP | IC₅₀ (μM) | IC₅₀ (μM) | DU145 |
| | IC₅₀ (μM) | AR-positive; | AR-positive; | IC₅₀ (μM) |
| Compound ID | AR-positive | AR-V7 positive | AR-V7 positive | AR-negative |
| Enzalutamide | 0.23 | >20 | >20 | >20 |
| Compound AA | 7.01 | >20 | >20 | 17.23 |
| Compound 2 | 0.52 | 2.75 | 8.81 | >20 |
| Compound 6 | 0.86 | 3.24 | | >20 |

### Example 13: AR Phase Separation Inhibition IC₅₀ Testing

Nuclear Puncta Imaging for Compounds IC₅₀ Determination
LNCaP cells were cultured in a 10-cm dish. Cells were transfected with AR (AR or AR F877L/T878A)-mEGFP expression plasmids (pMSCV vector, 12 μg plasmid per 10-cm dish). 12 hours after transfection, medium were replaced with steroid hormone-free and phenol red-free RPMI1640 (starvation medium) and continued to starve for another 24 hours. Cells (80 μl, 20,000 per well) were seeded in a 96-well glass bottom plate (Cellvis, P96-1.5H-N) and cultured for another 24 hours. Then cells were incubated with 10× serial-diluted compounds (10 l) for 3 hours before 20 nM DHT stimulation (10 l). After 3 hours DHT addition, cells were fixed with equal volume pre-warmed 8% PFA for 30 minutes and subsequently replaced with 100 μl PBS. Nuclear puncta images were acquired with Operetta CLS (Perkin Elmer) equipped with 60× water immersion lens (40 fields per well). The images were analyzed with Operetta built-in analysis modules. Nuclei were found from illumination corrected images with find nuclei module (type M) and filtered out low intensity nuclei. Subsequently puncta were first identified using find spot module (type A) and classified to high confident puncta and low confident puncta with PhenoLOGIC machine learning plug-in. For better representation, only high confident puncta were kept. The analyzed image puncta data were exported and statistics were processed with R/Bioconductor. The specific IC₅₀ values were calculated by R/drc package (four-parameter log-logistic function).

| | AR phase separation | |
|---|---|---|
| Compound No | inhibition IC₅₀ | |
| Compound AA | >20 | μM |
| Compound 6 | 3.6 | μM |
| Compound 60 | 2.5 | μM |

### Example 14: Characterization of One or More Compounds in CRPC In Vivo Models

Tumor growth was measured in male mice bearing LNCaP, 22Rv1 or VCaP tumors. Castration was performed when tumors reached ˜200 mm³ and dosing started when tumors regrew to 200 mm³. Tumor volume and body weight were monitored twice a week.

1. A method of treating an androgen receptor (AR)-associated disease or condition in a subject, comprising administering to the subject a therapeutically effective amount of an AR condensate modulator, wherein the AR condensate modulator modulates an AR condensate comprising at least AR.

2. The method of claim 1, wherein the AR is a wild type AR or an AR variant.

3. The method of claim 2, wherein the AR variant is resistant to at least one androgen-deprivation therapy.

4. The method of claim 2, wherein the AR variant comprises one or more mutations, optionally in ligand binding domain (LBD).

5. The method of claim 2, wherein the one or more mutations are found at the residues selected from the group consisting of L702, V716, V731, W742, H875, F877, T878, D880, L882, S889, D891, E894 μM896, E898, T919, wherein the numbering is relative to SEQ ID NO: 1.

6. The method of claim 4 or 5, wherein the one or more mutations are selected from the group consisting of L702H, V716M, V731M, W742L/C, H875Y/Q, F877L, T878A/S, D880E, L882I, S889G, D891H, E894K, M896V/T, E898G, T919S

7. The method of claim 2 or 3, wherein the AR variant lacks all or part of LBD.

8. The method of any of the preceding claims, wherein the AR condensate is a transcriptional condensate.

9. The method of claim 8, wherein the transcriptional AR condensate further comprises a DNA/RNA sequence, histone, cofactor, mediator, RNA polymerase, or any combination thereof.

10. The method of claim 9, wherein the DNA/RNA sequence comprises AR-regulated gene, the histone comprises K27 acetylated H3, the cofactor comprises an LXXLL motif-containing protein, the mediator comprises MED1, and/or the RNA polymerase comprises phosphorylated RNA pol II.

11. The method of any of the preceding claims, wherein the AR condensate modulator modulates formation, stability, or activity of the AR condensate.

12. The method of any of the preceding claims, wherein the AR condensate modulator is an AR condensate inhibitor.

13. The method of claim 12, wherein the AR condensate inhibitor decreases level of the AR condensate, optionally stimulated by DHT, as measured by live cell imaging.

14. The method of claim 13, wherein the level of AR condensate, optionally stimulated by DHT, is quantified based on variation of fluorescence intensity within cell nucleus.

15. The method of any of claims 13-14, wherein the AR condensate inhibitor decreases the level of AR condensate, optionally stimulated by DHT, by at least 10%, 20%, 30%, 40%, 50%, 60%, 70%, or 80%.

16. The method of any one of claims 12-15, wherein the AR condensate inhibitor decrease expression level of at least one (or at least two, three, or four) AR-regulated gene as measured by a quantitative PCR assay in a AR-expressing cell line.

17. The method of claim 16, wherein the AR-regulated gene is an AR target gene or an AR variant target gene.

18. The method of claim 17, wherein the AR target gene is selected from the group consisting of FKBP5, KLK2, PSA, TMPRSS2, and NKX3.1

19. The method of claim 17, wherein the AR variant target gene is selected from the group consisting of BUB1B, CCNA2, UBE2C, KIF15, and CDC20.

20. The method of any of claims 16-19, wherein the AR condensate inhibitor is at least 2-fold (e.g. 3-fold, 4-fold, 5-fold, 6-fold etc.) more potent than enzalutamide at a comparable concentration in decreasing the expression level of the at least one (or at least two, three, or four) AR-regulated gene.

21. The method of any of claims 16-20, wherein the AR condensate inhibitor at a concentration of 5 μM decrease the expression level of the at least one (or at least two, three, or four) AR-regulated gene by at least 50%.

22. The method of any of claims 16-21, wherein the expression level of the at least one (or at least two, three, or four) AR-regulated gene is decreased by enzalutamide at a concentration of 5 μM by no more than 20%.

23. The method of any one of claims 12-22, wherein the AR condensate inhibitor inhibits proliferation of AR-expressing cancer cells at an IC₅₀ of no more than 20 μM, or 15 μM, or 10 μM or 8 μM or 5 μM or 4 μM or 3 μM, as measured by a cell proliferation assay.

24. The method of claim 23, wherein the AR-expressing cancer cells are selected from the group consisting of LNCaP, 22Rv1, VCaP and LNCaP95.

25. The method of any of claims 12-24, wherein the AR condensate modulator:
a) comprises Compound 60, Compound 61, Compound 62, Compound 6, Compound 2, or a pharmaceutically acceptable salt, solvate, prodrug, enantiomer, diastereomer, tautomer, isotopic substitution, polymorph or metabolite thereof, or
b) competes with Compound 60, Compound 61, Compound 62, Compound 6, or Compound 2 for binding to AR, or
c) induces a conformational change in AR at least comparable to that induced by Compound 60, Compound 61, Compound 62, Compound 6, or Compound 2;
d) has an activity comparable to or higher than that of Compound 60, Compound 61, Compound 62, Compound 6, or Compound 2 in any one or any combination of the following:
i) decreasing level of the AR condensate, optionally stimulated by DHT;
ii) decreasing expression level of at least one (or at least two, three, or four) AR-regulated gene; and/or
iii) inhibiting proliferation of AR-expressing cancer cells.

26. The method of any one of preceding claims, wherein the AR condensate modulator binds to the intrinsic disorder domain (IDD) of AR.

27. The method of any of the preceding claims, wherein the AR condensate modulator comprises a peptide, nucleic acid, or small molecule.

28. The method of any of the preceding claims, wherein the AR-associated disease or condition is characterized in having an abnormal level of AR activity.

29. The method of any of the preceding claims, wherein the AR-associated disease or condition is characterized in having an elevated level of AR activity.

30. The method of any of the preceding claims, wherein the AR-associated disease or condition is characterized having one or more genetic aberrations that results in elevated AR activity, constitutive AR activation, or resistance to castration or androgen deprivation therapy.

31. The method of claim 30, wherein the one or more genetic aberrations comprise amplification of AR gene, mutations in AR gene, aberrant splicing of AR gene, rearrangement in AR gene, polymorphism in AR gene, or any combination thereof.

32. The method of any of the preceding claims, wherein the AR-associated disease or condition is characterized having an AR variant, optionally, the AR variant lacks all or part of the ligand binding domain (LBD).

33. The method of any of the preceding claims, wherein the AR-associated disease or condition is an AR-expressing cancer.

34. The method of claim 33, wherein the AR-expressing cancer is prostate cancer, breast cancer, glioblastoma, melanoma, bladder cancer, renal cell carcinoma, pancreatic cancer, hepatocellular carcinoma, ovarian cancer, endometrial cancer, mantle cell lymphoma, or salivary gland cancer.

35. The method of any of claims 33-34, wherein the AR-expressing cancer is metastatic.

36. The method of any of claims 33-35, wherein the AR-expressing cancer is prostate cancer.

37. The method of claim 36, wherein the prostate cancer is resistant to castration or androgen deprivation therapy.

38. A method of modulating transcription of one or more AR-regulated genes in a cell or a subject, comprising modulating a transcriptional AR condensate comprising at least AR.

39. The method of claim 38, wherein the AR is a wild type AR or an AR variant.

40. The method of claim 38 or 39, comprising modulating the transcriptional AR condensate by an AR condensate modulator.

41. The method of claim 40, wherein the AR condensate modulator modulates formation, stability, or activity of the AR condensate.

42. The method of any one of claims 38-41, wherein the AR condensate modulator is an AR condensate inhibitor.

43. The method of any one of claims 38-42, wherein the AR-regulated gene is an AR target gene or an AR variant target gene.

44. The method of claim 43, wherein the AR target gene is selected from the group consisting of FKBP5, KLK2, PSA, TMPRSS2, and NKX3.1.

45. The method of claim 43, Wherein the AR variant target gene is selected from the group consisting of BUB1B, CCNA2, UBE2C, KIF15, and CDC20.

46. The method of any one of claims 38-45, wherein the AR condensate modulator:
a) comprises Compound 60, Compound 61, Compound 62, Compound 6, or Compound 2, or a pharmaceutically acceptable salt, solvate, prodrug, enantiomer, diastereomer, tautomer, isotopic substitution, polymorph or metabolite thereof, or
b) competes with Compound 60, Compound 61, Compound 62, Compound 6, or Compound 2 for binding to AR, or
c) induces a conformational change in AR at least comparable to that induced by Compound 60, Compound 61, Compound 62, Compound 6, or Compound 2;
d) has an activity comparable to or higher than that of Compound 60, Compound 61, Compound 62, Compound 6, or Compound 2 in any one or any combination of the following:
i) decreasing level of the AR condensate, optionally stimulated by DHT;
ii) decreasing expression level of at least one (or at least two, three, or four) AR-regulated gene; and/or
iii) inhibiting proliferation of AR-expressing cancer cells.

47. The method of any of claims 38-46, wherein the AR condensate inhibitor binds to the intrinsic disorder domain (IDD) of AR.

48. The method of any of claims 38-47, wherein the AR condensate modulator comprises a peptide, nucleic acid, or small molecule.

49. The method of any of claims 38-48, wherein the cell or subject is characterized in having an abnormal level of AR activity, or an elevated level of AR activity.

50. The method of claim 49, wherein the cell or subject is characterized in having one or more genetic aberrations that results in elevated AR activity, constitutive AR activation, or resistance to castration or androgen deprivation therapy.

51. The method of claim 50, wherein the one or more genetic aberrations comprise amplification of AR gene, mutations in AR gene, aberrant splicing of AR gene, rearrangement in AR gene, polymorphism in AR gene, or any combination thereof.

52. The method of claim 51, wherein the cell or subject is characterized in having an AR variant, optionally, the AR variant lacks all or part of the ligand binding domain (LBD).

53. An in vitro screening system comprising AR or an intrinsic disorder domain containing fragment thereof, wherein the AR or the fragment is attached to a detectable label and is capable of forming an AR condensate.

54. The in vitro screening system of claim 53, wherein the AR is a wild type AR or an AR variant.

55. The in vitro screening system of claim 53 or 54, wherein the detectable label comprises a fluorophore, a radioisotope, a colorimetric substrate, or an antigenic epitope.

56. The in vitro screening system of any of claims 53-55, wherein the in vitro screening system comprises a cell lysate or a nuclear lysate.

57. The in vitro screening system of any of claims 53-55, wherein the in vitro screening system comprises a cell or a nucleus.

58. The in vitro screening system of any of claims 53-57, wherein the in vitro screening system further comprises a DNA/RNA sequence, histone, cofactor, mediator, RNA polymerase, or any combination thereof.

59. A synthetic AR condensate comprising at least AR or a fragment thereof comprising IDD.

60. A modified host cell expressing AR or an intrinsic disorder domain containing fragment thereof, wherein the AR or the fragment is attached to a detectable label and is capable of forming an AR condensate.

61. The modified host cell of claim 60, wherein the AR is a wild type AR or an AR variant.

62. The modified host cell of claim 60 or 61, wherein the detectable label comprises a fluorophore, a radioisotope, a colorimetric substrate, or an antigenic epitope.

63. The modified host cell of any of claims 60-62, which is suitable for detection of formation of the AR condensate.

64. The modified host cell of any of claims 60-63, wherein the host cell is a tumor cell, optionally a prostate cancer cell.

65. A method of screening for an agent that modulates an AR condensate comprising at least AR, comprising:
a. providing the AR condensate, and assessing one or more physical properties or one or more biological effects of the AR condensate,
b. contacting the AR condensate with a test agent, and
c. assessing whether the test agent causes a change in the one or more physical properties or one or more biological effects of the AR condensate.

66. The method of claim 65, wherein the test agent is identified as modulating the condensate if it causes a change in the one or more physical properties or one or more biological effects of the AR condensate.

67. The method of claim 65 or 66, wherein the physical properties comprises formation, composition, stability, and/or activity of the AR condensate.

68. The method of claim any of claims 65-67, wherein the AR condensate is contained in an in vitro screening system of any of claims 53-58, a synthetic AR condensate of claim 59, or in a modified host cell of any of claims 60-64.

69. The method of any of claims 65-67, wherein the AR condensate is an isolated synthetic condensate, or is in the form of an isolated cellular composition comprising the AR condensate.

70. The method of any of claims 65-67, wherein the AR condensate is inside a cell or inside nucleus.

71. A method of identifying an agent that modulates formation of an AR condensate comprising at least AR, comprising:
a. providing components capable of forming the AR condensate;
b. contacting the components with a test agent under the condition suitable for formation of the AR condensate, and
c. assessing whether presence of the test agent affects formation of the AR condensate or one or more biological effects of the AR condensate.

72. The method of claim 71, wherein the test agent is identified as modulating the formation of the condensate if it affects formation of the AR condensate or affects the one or more biological effects of the AR condensate.

73. The method of claim 71, wherein the components capable of forming the AR condensate are contained in an in vitro screening system of any of claims 53-58, a synthetic AR condensate of claim 59, or in a modified host cell of any of claims 60-64.

74. The method of any of claims 71-73, wherein the AR condensate is a transcriptional condensate.

75. The method of any of claims 71-74, wherein the one or more biological effects of the transcriptional condensate is assessed based on expression of an AR-regulated gene or cell proliferation.

76. The method of claim 75, wherein the AR-regulated gene is an AR target gene, an AR variant target gene, or a reporter gene.

77. The method of any of claims 71-76, wherein the test agent has been identified as capable of binding to IDD of AR.

78. A compound comprising a chemical structure selected from the group consisting of: 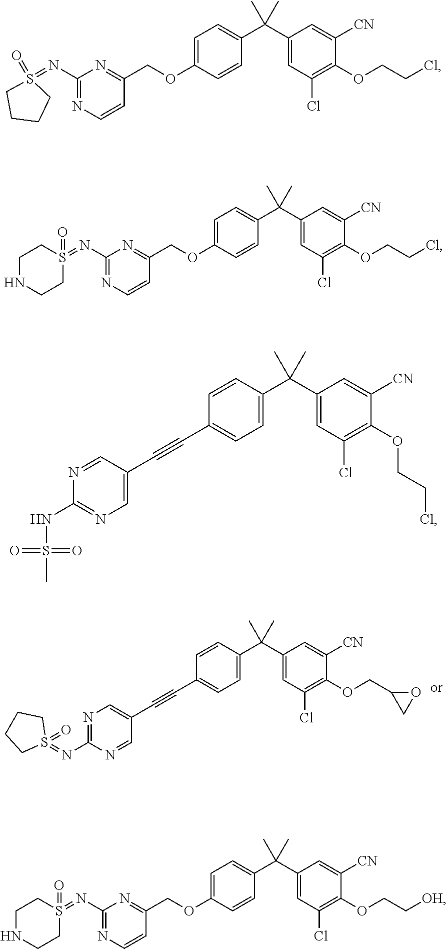 [SMILES: CC(C)(c1ccc(OCc2ccnc(N=S3(=O)CCCC3)n2)cc1)c1cc(Cl)c(OCCCl)c(C#N)c1 ; CC(C)(c1ccc(OCc2ccnc(N=S3(=O)CCNCC3)n2)cc1)c1cc(Cl)c(OCCCl)c(C#N)c1 ; CC(C)(c1ccc(C#Cc2cnc(NS(C)(=O)=O)nc2)cc1)c1cc(Cl)c(OCCCl)c(C#N)c1 ; CC(C)(c1ccc(C#Cc2cnc(N=S3(=O)CCCC3)nc2)cc1)c1cc(Cl)c(OCC2CO2)c(C#N)c1 ; CC(C)(c1ccc(OCc2ccnc(N=S3(=O)CCNCC3)n2)cc1)c1cc(Cl)c(OCCO)c(C#N)c1]
or a pharmaceutically acceptable salt, solvate, prodrug, enantiomer, diastereomer, tautomer, isotopic substitution, polymorph or metabolite thereof.

79. A pharmaceutical composition comprising a compound selected from the group consisting of:
3-chloro-2-(2-chloroethoxy)-5-(2-(4-((2-((1-oxidotetrahydro-1λ⁶-thiophen-1-ylidene) amino) pyrimidin-4-yl) methoxy) phenyl) propan-2-yl) benzonitrile,
3-chloro-2-(2-chloroethoxy)-5-(2-(4-((2-((1-oxido-1λ⁶-thiomorpholin-1-ylidene) amino)pyrimidin-4-yl)methoxy)phenyl)propan-2-yl)benzonitrile,
N-(5-((4-(2-(3-chloro-4-(2-chloroethoxy)-5-cyanophenyl)propan-2-yl)phenyl)ethynyl)pyrimidin-2-yl)methanesulfonamide,
3-chloro-5-(2-(4-((2-((1-oxidotetrahydro-1λ⁶-thiophen-1-ylidene)amino)pyrimidin-5-yl)ethynyl)phenyl)propan-2-yl)-2-(oxiran-2-ylmethoxy)benzonitrile, and
3-chloro-2-(2-hydroxyethoxy)-5-(2-(4-((2-((1-oxido-1λ⁶-thiomorpholin-1-ylidene)amino)pyrimidin-4-yl)methoxy)phenyl)propan-2-yl)benzonitrile, or a pharmaceutically acceptable salt, solvate, prodrug, enantiomer, diastereomer, tautomer, isotopic substitution, polymorph or metabolite thereof.

80. A method of treating an AR-associated disease or condition in a subject, comprising administering to the subject a therapeutically effective amount of a compound selected from the group consisting of:
3-chloro-2-(2-chloroethoxy)-5-(2-(4-((2-((1-oxidotetrahydro-1λ⁶-thiophen-1-ylidene) amino) pyrimidin-4-yl) methoxy) phenyl) propan-2-yl) benzonitrile,
3-chloro-2-(2-chloroethoxy)-5-(2-(4-((2-((1-oxido-1λ⁶-thiomorpholin-1-ylidene) amino)pyrimidin-4-yl)methoxy)phenyl)propan-2-yl)benzonitrile,
N-(5-((4-(2-(3-chloro-4-(2-chloroethoxy)-5-cyanophenyl)propan-2-yl)phenyl)ethynyl)pyrimidin-2-yl)methanesulfonamide,
3-chloro-5-(2-(4-((2-((1-oxidotetrahydro-1λ⁶-thiophen-1-ylidene)amino)pyrimidin-5-yl)ethynyl)phenyl)propan-2-yl)-2-(oxiran-2-ylmethoxy)benzonitrile, or
3-chloro-2-(2-hydroxyethoxy)-5-(2-(4-((2-((1-oxido-1λ⁶-thiomorpholin-1-ylidene)amino)pyrimidin-4-yl)methoxy)phenyl)propan-2-yl)benzonitrile, or a pharmaceutically acceptable salt, solvate, prodrug, enantiomer, diastereomer, tautomer, isotopic substitution, polymorph or metabolite thereof.

81. The method of claim 80, wherein the compound is administered in combination with a second active ingredient or therapy.

82. The method of claim 81, wherein the second active ingredient or therapy is an anti-cancer therapy, or optionally, an anti-prostate cancer drug.
